(19) United States
(12) Patent Application Publication
(10) Pub. No.: US 2024/0335400 A1
(43) Pub. Date: Oct. 10, 2024

(54) GASTRIC RESIDENCE SYSTEMS COMPRISING METHADONE

(71) Applicant: Lyndra Therapeutics, Inc., Watertown, MA (US)

(72) Inventors: Estelle BEGUIN, Watertown, MA (US); Patricia QUARTON, Watertown, MA (US); Jeffrey KATSTRA, Watertown, MA (US); David ALTREUTER, Watertown, MA (US); Juan Jaramillo MONTEZCO, Cambridge, MA (US); Manjeet PIMPARADE, Watertown, MA (US)

(21) Appl. No.: 18/289,326

(22) PCT Filed: May 4, 2022
(86) PCT No.: PCT/US2022/072115

§ 371 Date: Nov. 2, 2023

Related U.S. Application Data
(60) Provisional application No. 63/184,760, filed on May 5, 2021.

(51) Int. Cl. A61K 31/137; A61K 9/00; A61K 47/34

(52) U.S. Cl. CPC A61K 31/137; A61K 9/0065; A61K 47/34

(57) ABSTRACT

Provided are gastric residence systems comprising at least one drug-eluting component comprising methadone or a salt thereof, 35-50 wt % polycaprolactone, and 0.5-3 wt % poloxamer, and a rate-modulating release film coating the at least one co-extruded drug eluting component. The gastric residence systems comprising at least one drug-eluting component are configured to be maintained within a stomach of a human body for at least 48 hours and to release methadone for at least 48 hours, such that the at least one drug eluting component with the rate-modulating release film is configured to release at least 10% of the methadone or the salt thereof after the first 24 hours of residence within the stomach.

### CROSS-REFERENCE TO RELATED APPLICATIONS

This application is a national stage application under 35 U.S.C. § 371 of International Application No. PCT/US2022/072115, filed internationally on May 4, 2022, which claims the benefit of U.S. Provisional Application No. 63/184,760 filed May 5, 2021, the disclosures of which are hereby incorporated herein by reference in their entirety.

### STATEMENT REGARDING FEDERALLY SPONSORED RESEARCH OR DEVELOPMENT

This invention was made with government support under Grant No. 1UG3DA050310 awarded by the National Institutes of Health. The government has certain rights in the invention.

### FIELD

The present disclosure relates to gastric residence systems, and more particularly, to gastric residence systems comprising methadone.

### BACKGROUND

Methadone is an opioid and μOR agonist approved for opioid-use disorder maintenance therapy. Methadone lessens opiate withdrawal symptoms and blocks the euphoric effects of opiate drugs such as heroin, morphine, and codeine, as well as semi-synthetic opioids like oxycodone and hydrocodone. Methadone maintenance therapy (MMT) has been employed since the 1960s and has been shown to facilitate recovery and prevent deaths.

Because methadone is a full μOR agonist, it carries significant risks of accidental overdose and diversion. Thus, methadone is usually administered on a once-daily schedule under direct observation at a designated clinic site. After methadone administration at a designated clinic site, a patient may be observed for 15-20 minutes to reduce risk of medication diversion.

### SUMMARY OF THE DISCLOSURE

Provided herein are gastric residence systems comprising methadone. Currently, patients receiving methadone to treat opioid-use disorder usually take a daily dose of methadone at a designated clinic site. Thus, to properly follow a treatment plan, the patient must travel to a designated clinic site each day to receive their daily dose of methadone. As expected, this can be difficult for many patients and can easily lead to non-compliance.

The gastric residence systems comprising methadone provide herein are designed to be administered to a patient less frequently than conventional maintenance therapy methods. As explained above, methadone is typically administered to a patient on a daily basis in a supervised setting. However, this is very burdensome not only to the patient, who must travel to and from the designated clinic site each day, but also to the designated clinic site, which must have the proper medication dosages and staffing available each day to handle its patient. Accordingly, the gastric residence systems provided herein are designed to be administered less frequently than once a day, yet still provide the same therapeutic benefits as a daily methadone maintenance therapy. For example, gastric residence systems provided herein may be configured to be administered once a week. Throughout the residence period (e.g., 7 days), the gastric residence system is configured to slowly release methadone within the patient's stomach. A less frequent dosage administration, lessens the burden on patients who must travel to a designated clinic site each day and the burden on designated clinic sites who must accommodate the patients each day during daily methadone maintenance therapy. Thus, the gastric residence systems provided herein, which need to be administered at a frequency less than that of daily methadone maintenance therapy, can minimize non-compliance among patients and can lessen the amount of resources required by designated clinic sites.

The gastric residence systems provided herein can achieve a steady methadone release throughout the residence period of the gastric residence system in a patient's stomach only with the combination of the methadone formulation in a drug-eluting segment of a gastric residence system and a release rate-modulating film coating the drug-eluting segments of the gastric residence system. Without both of these features, the gastric residence system will not properly release methadone in a controlled manner throughout the residence period. For example, if a gastric residence system is configured to have a residence period of 7 days, the methadone formulation combined with the release rate-modulating film coating can control the methadone release such that the methadone continues to release from the gastric residence system throughout the residence period, including on day 7 (i.e., the last day) of the residence period.

The gastric residence systems provided are designed to be swallowed by a patient in a compacted configuration, and, once arriving at the patient's stomach, open to an uncompacted configuration. Once open in the patient's stomach, the gastric residence system will remain in the stomach until the gastric fluids of the stomach cause the gastric residence system to breakdown such that it can pass through the remainder of the patient's gastrointestinal system. The gastric residence systems can comprise one or more linking components (e.g., linkers) linking various components of the gastric residence system together. The linking components comprise a disintegrating matrix and are designed to dissolve or breakdown in the presence of gastric fluids in a controlled manner, allowing various components of the gastric residence system to disassociate and pass through the rest of the patient's gastrointestinal tract. In some embodiments, the gastric residence systems are designed to release the active ingredients (i.e., methadone), dissociate, and exit the patient in a controlled amount of time (e.g., 12 hours, 1 day, 2 days, 3 days, 4 days, 5 days, 6 days, 7 days, or 1 month) after the patient first ingests the gastric residence system.

In some embodiments, the gastric residence systems are in the shape of a stellate and comprise a central elastomer, three or more arms, and one or more linking components. The central elastomer of the gastric residence system is the center component of the gastric residence system, from which the three or more arms extend.

The three or more arms of the gastric residence system extend radially outward from the central elastomer of the gastric residence system. One or more of the arms may include the active pharmaceutical ingredients (i.e., methadone). The number of active arms (i.e., the one or more arms comprising the active pharmaceutical ingredients) may depend on the dosage amount of the gastric residence system. The active arms are configured to release the methadone formulation once the gastric residence system reaches the patient's stomach.

Accordingly, provided herein are gastric residence systems comprising methadone for use in treating patients having opioid use disorder. The gastric residence systems described are designed to be administered to a patient at a lower frequency than that of conventional methadone maintenance therapies. In some embodiments, the gastric residence systems provided herein are designed to be administered to a patient once every 2, 3, 4, 5, 6, 7, 8, 9, 10, 11, 12, 13, or 14 days. In some embodiments, the gastric residence systems provided herein are designed to be administered to a patient once every 1, 2, 3, 4, 5, 6, 7, or 8 weeks.

In some embodiments, a gastric residence system is provided, the gastric residence system comprising: at least one drug-eluting component comprising methadone or a salt thereof, 35-50 wt % polycaprolactone, and 0.5-3 wt % poloxamer; and a release rate-modulating film coating the at least one drug-eluting component, wherein the gastric residence system is configured to be maintained within a stomach of a human body for at least 48 hours and to release methadone for at least 48 hours, and the at least one drug-eluting component with the release rate-modulating film is configured to release at least 10% of the methadone or the salt thereof after the first 24 hours of residence within the stomach.

In some embodiments of the gastric residence system, the gastric residence system comprises 50-60 wt % racemic methadone.

In some embodiments of the gastric residence system, the gastric residence system comprises 50-60 wt % levomethadone.

In some embodiments of the gastric residence system, the gastric residence system comprises 1-2 wt % poloxamer.

In some embodiments of the gastric residence system, the poloxamer comprises P407.

In some embodiments of the gastric residence system, the release rate-modulating film comprises polycaprolactone, copovidone, and magnesium stearate.

In some embodiments of the gastric residence system, the release rate-modulating film comprises 60-90 wt % polycaprolactone.

In some embodiments of the gastric residence system, the release rate-modulating film comprises 70-75 wt % polycaprolactone.

In some embodiments of the gastric residence system, the release rate-modulating film comprises 10-40 wt % copovidone.

In some embodiments of the gastric residence system, the release rate-modulating film comprises 20-30 wt % copovidone.

In some embodiments of the gastric residence system, the release rate-modulating film comprises 1-5 wt % magnesium stearate.

In some embodiments of the gastric residence system, the release rate-modulating film comprises 1-3 wt % magnesium stearate.

In some embodiments of the gastric residence system, the at least one drug-eluting component comprises 20 mg to 50 mg of racemic methadone or a salt thereof.

In some embodiments of the gastric residence system, the at least one drug-eluting component comprises 20 mg to 50 mg of levomethadone or a salt thereof.

In some embodiments of the gastric residence system, the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

In some embodiments of the gastric residence system, the plurality of arms comprises six arms.

In some embodiments of the gastric residence system, at least two arms of the plurality of arms comprises a drug-eluting component of the at least one drug-eluting component.

In some embodiments of the gastric residence system, at least three arms of the plurality of arms comprises a drug-eluting component of the at least one drug-eluting component.

In some embodiments of the gastric residence system, six arms of the plurality of arms comprises a drug-eluting component of the at least one drug-eluting component.

In some embodiments of the gastric residence system, each arm of the plurality of arms comprises a polymeric linker segment attached to the central elastomer, the polymeric linker segment comprising polycaprolactone.

In some embodiments of the gastric residence system, each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

In some embodiments of the gastric residence system, each arm of the plurality of arms comprises a first inert segment attached to the first disintegrating matrix segment, the first inert segment comprising polycaprolactone and (BiO)2CO3.

In some embodiments of the gastric residence system, each arm of the plurality of arms comprises a second disintegrating matrix segment attached to the first inert segment, the second disintegrating matrix segment comprising polycaprolactone, hydroxypropyl methylcellulose acetate succinate, and a poloxamer.

In some embodiments of the gastric residence system, each arm of the plurality of arms comprises an inactive segment attached to the second disintegrating matrix segment, the inactive segment comprising polycaprolactone, copovidone, and a poloxamer.

In some embodiments of the gastric residence system, a drug-eluting arm of the plurality of arms comprises the drug-eluting component attached to the inactive segment.

In some embodiments of the gastric residence system, the area under the curve of the gastric residence system is between 1000 and 6000 hr·ng/mL.

In some embodiments, a method of treating an opioid abuse disorder in an individual is provided, the method comprising administering the gastric residence system to the individual.

In some embodiments, a method of making a gastric residence system is provided, the method comprising: extruding at least one drug-eluting component comprising methadone or a salt thereof, 35-50 wt % polycaprolactone, and 0.5-3 wt % poloxamer; and applying a release rate-modulating film to the at least one drug-eluting component, wherein the gastric residence system is configured to be maintained within a stomach of a human body for at least 48 hours and to release methadone for at least 48 hours, and the at least one drug-eluting component with the release rate-modulating film is configured to release at least 10% of the methadone or the salt thereof after the first 24 hours of residence within the stomach.

In some embodiments of the method, the gastric residence comprises 50-60 wt % racemic methadone.

In some embodiments of the method, the gastric residence comprises 50-60 wt % levomethadone.

In some embodiments of the method, the gastric residence comprises 1-2 wt % poloxamer.

In some embodiments of the method, the poloxamer comprises P407.

In some embodiments of the method, the release rate-modulating film comprises polycaprolactone, copovidone, and magnesium stearate.

In some embodiments of the method, the release rate-modulating film comprises 60-90 wt % polycaprolactone.

In some embodiments of the method, the release rate-modulating film comprises 70-75 wt % polycaprolactone.

In some embodiments of the method, the release rate-modulating film comprises 10-40 wt % copovidone.

In some embodiments of the method, the release rate-modulating film comprises 20-30 wt % copovidone.

In some embodiments of the method, the release rate-modulating film comprises 1-5 wt % magnesium stearate.

In some embodiments of the method, the release rate-modulating film comprises 1-3 wt % magnesium stearate.

In some embodiments of the method, the at least one drug-eluting component comprises 20 mg to 50 mg of racemic methadone or a salt thereof.

In some embodiments of the method, the at least one drug-eluting component comprises 20 mg to 50 mg of levomethadone or a salt thereof.

In some embodiments of the method, the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

In some embodiments of the method, the plurality of arms comprises six arms.

In some embodiments of the method, at least two arms of the plurality of arms comprises a drug-eluting component of the at least one drug-eluting component.

In some embodiments of the method, at least three arms of the plurality of arms comprises a drug-eluting component of the at least one drug-eluting component.

In some embodiments of the method, six arms of the plurality of arms comprises a drug-eluting component of the at least one drug-eluting component.

In some embodiments of the method, each arm of the plurality of arms comprises a polymeric linker segment attached to the central elastomer, the polymeric linker segment comprising polycaprolactone.

In some embodiments of the method, each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

In some embodiments of the method, each arm of the plurality of arms comprises a first inert segment attached to the first disintegrating matrix segment, the first inert segment comprising polycaprolactone and (BiO)2CO3.

In some embodiments of the method, each arm of the plurality of arms comprises a second disintegrating matrix segment attached to the first inert segment, the second disintegrating matrix segment comprising polycaprolactone, hydroxypropyl methylcellulose acetate succinate, and a poloxamer.

In some embodiments of the method, each arm of the plurality of arms comprises an inactive segment attached to the second disintegrating matrix segment, the inactive segment comprising polycaprolactone, copovidone, and a poloxamer.

In some embodiments of the method, a drug-eluting arm of the plurality of arms comprises the drug-eluting component attached to the inactive segment.

In some embodiments of the method, the area under the curve of the gastric residence system is between 1000 and 6000 hr·ng/mL.

In some embodiments, a gastric residence system is provided, the gastric residence system comprising: a plurality of arms affixed to a central elastomer, wherein at least one arm comprises a drug-eluting component; each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein the at least one arm comprising a drug eluting component comprises: a polymeric linker segment; a first disintegrating matrix segment attached to the polymeric linker segment; a first inert segment attached to the first disintegrating matrix segment; a second disintegrating matrix segment attached to the first inert segment; an inactive segment attached to the second disintegrating matrix segment; and the drug-eluting component attached to the inactive segment, wherein the drug eluting component comprises 50-60 wt % methadone or a salt thereof, 35-50 wt % polycaprolactone, and 0.5-3 wt % poloxamer, and wherein the drug eluting component further comprises a coating comprising a release rate-modulating polymer film.

In some embodiments, any one or more of the features, characteristics, or elements discussed above with respect to any of the embodiments may be incorporated into any of the other embodiments mentioned above or described elsewhere herein.

### BRIEF DESCRIPTION OF THE FIGURES

This application contains at least one drawing executed in color. Copies of this patent application publication with color drawing(s) will be provided by the Office upon request and payment of the necessary fee.

FIG. 1A shows a gastric residence system having a stellate configuration, according to some embodiments;

FIG. 1B shows a gastric residence system in a folded configuration, according to some embodiments;

FIG. 2 shows the arrangement of elongate arms of a gastric residence system with six elongate arms, where the arm cross-section is wedge-shaped, according to some embodiments;

FIG. 3 shows a gastric residence system having six active arms, according to some embodiments;

FIG. 4 shows a gastric residence system having four active arms, according to some embodiments;

FIG. 5A shows methadone release profiles for coated and uncoated drug-eluting segments;

FIG. 5B shows methadone release profiles for coated and uncoated drug-eluting segments;

FIG. 6 shows methadone release profiles for gastric residence systems comprising drug-eluting segments coated with various amounts of a release rate-modulating film;

FIG. 7 shows methadone release rates for drug-eluting segments in various simulated fluids (i.e., FaSSGF, FED, FaSSIF);

FIGS. 8A and 8B show methadone release for a 1-day ethanol challenge;

FIG. 9A and 9B show methadone release for a 3-day ethanol challenge;

FIG. 10A shows the architecture of an active arm of a gastric residence system used in Example 6;

FIG. 10B shows mean methadone plasma concentrations over 10 days following a single oral dose of LYN-014-M (100 mg);

FIG. 10C shows mean methadone plasma concentrations following a single oral dose of methadone HCl (10 mg);

FIG. 10D shows mean methadone plasma concentrations over 7 days following a single oral dose of LYN-014-M (100 mg);

FIG. 11 shows a cumulative release profile using a dissolution testing method; and

FIG. 12 shows a cumulative release profile using an in vitro release testing method.

### DETAILED DESCRIPTION OF THE DISCLOSURE

Described herein are gastric residence systems comprising methadone. In some embodiments, gastric residence systems comprising methadone provided herein may be used to treat opioid-use disorder. In some embodiments, the gastric residence systems described are designed to be administered at a lower frequency than conventional methadone dosage forms. By decreasing the administration frequency, dosage forms comprising methadone provided herein may increase patient compliance. For example, instead of a patient having to travel to a designated clinic site once a day, as is typically required of conventional methadone maintenance therapy plans, a patient would only have to travel to a designated clinic site once every other day, once a week, or once a month, for example. (A patient is usually administered methadone in a clinical setting to minimize the incidence of overdose or diversion.) This not only lessens the burden placed on the patient, since they won't have to travel to the designated clinic site as frequently, but less frequent administration also lessens the burden on designated clinic sites that must accommodate regular visits from patients. To achieve a lower dosage frequency, the gastric residence systems provided herein slowly release the active pharmaceutical ingredient(s) (i.e., methadone) over the course of the residence period. Thus, if the gastric residence form is designed to have a residence period of one week, then the active pharmaceutical ingredient(s) (i.e., methadone) is designed to slowly release in the patient's stomach throughout the one-week residence period.

The specific combination of the methadone formulation for a drug-eluting segment of a gastric residence system described herein, in combination with a release rate-modulating film described herein that can achieve a gastric residence system that needs to be administered to a patient at a frequency that is less than that of conventional methadone maintenance therapies. Without both of these features, the gastric residence system will not properly release methadone in a controlled manner throughout the residence period. For example, if a gastric residence system is configured to have a residence period of 7 days, the methadone formulation combined with the release rate-modulating film coating can control the methadone release such that the methadone continues to release from the gastric residence system throughout the residence period, including on day 7 (i.e., the last day) of the residence period.

The gastric residence systems for administering methadone may be in the shape of a stellate. For example, the stellate-shaped gastric residence systems may include a central elastomer, three or more arms, and a plurality of linking components (i.e., linkers). The central elastomer is located at the center of the stellate, and the three or more arms may extend radially from the central elastomer. The linkers may couple two or more components of the gastric residence systems together. For example, a linker may connect an arm to the central elastomer. A linker might also connect two lengths of an arm together (e.g., act as an elbow of the arm). In some embodiments, the active pharmaceutical ingredient (i.e., the methadone) may be provided in an arm of the stellate (i.e., an active arm). The active arm(s) may include a methadone formulation.

Described below are gastric residence systems for administering methadone to a patient. Specifically, the discussion below provides: (1) definitions; (2) gastric residence system drug delivery mechanism; (3) configuration and components of gastric residence systems; (4) features for improved retention and agent release; (5) carrier polymer-agent segments comprising a methadone formulation; (6) rate-modulating polymer films; (7) gastric residence time; (8) gastric residence systems comprising methadone; and (9) examples.

### Definitions

“Methadone” may be racemic or levomethadone. Levomethadone is approximately twice as potent as racemic methadone. “Methadone” may also refer to a salt of methadone, such as methadone HCl or levomethadone HCl.

A “carrier polymer” is a polymer suitable for blending with an agent, such as a drug, for use in a gastric residence system.

An “agent” is any substance intended for therapeutic, diagnostic, or nutritional use in a patient, individual, or subject. Agents include, but are not limited to, drugs, nutrients, vitamins, and minerals.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer” or “elastomer” is a polymer that is capable of being deformed by an applied force from its original shape for a period of time, and which then substantially returns to its original shape once the applied force is removed.

A “patient,” “individual,” or “subject” refers to a mammal, preferably a human or a domestic animal such as a dog or cat. In a most preferred embodiment, a patient, individual, or subject is a human.

A “poloxamer” is a block co-polymer having a central polypropylene oxide core, with a flanking region of polyethylene oxide on either side of the core.

The “diameter” of a particle as used herein refers to the longest dimension of a particle.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods disclosed herein can then be used to treat asymptomatic patients at risk of developing the clinical symptoms of the disease or disorder, in order to suppress the appearance of any adverse symptoms.

A “flexural modulus” of a material is an intrinsic property of a material computed as the ratio of stress to strain in flexural deformation of the material as measured by a 3-point bending test. Although the linkers are described herein as being components of the gastric residence system, the flexural modulus of the material of the polymeric material may be measured in isolation. For example, the polymeric linker in the gastric residence system may be too short to measure the flexural modulus, but a longer sample of the same material may be used to accurately determine the flexural modulus. The longer sample used to measure the flexural modulus should have the same cross-sectional dimensions (shape and size) as the polymeric linker used in the gastric residence system. The flexural modulus is measured using a 3-point bending test in accordance with the ASTM standard 3-point bending test (ASTM D790) using a 10 mm distance between supports and further modified to accommodate materials with non-rectangular cross-sections. The longest line of symmetry for the cross section of the polymeric linker should be positioned vertically, and the flexural modulus should be measured by applying force downward. If the longest line of symmetry for the cross section of the polymeric linker is perpendicular to a single flat edge, the single flat edge should be positioned upward. If the cross-section of the polymeric linker is triangular, the apex of the triangle should be faced downward. As force is applied downward, force and displacement are measured, and the slope at the linear region is obtained to calculate the flexural modulus.

As used herein, the singular forms “a”, “an”, and “the” include plural references unless indicated otherwise or the context clearly dictates otherwise.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present disclosure.

In addition to the embodiments and methods disclosed here, additional embodiments of gastric residence systems, and methods of making and using such systems, are disclosed in International Patent Application Nos. WO 2015/191920, WO 2015/191925, WO 2017/070612, WO 2017/100367, and PCT/US2017/034856, which are incorporated by reference herein in their entirety.

The following abbreviations for polymers and other components are used:

| Abbreviation | Component |
|---|---|
| PDL | poly(DL-lactide); inherent viscosity 1.6-2.4 dl/g (CHCl₃), Tₘ |
| | 165-180° C. |
| PCL | polycaprolactone |
| VA64 | copovidone; Tₘ 140° C., T_{g} 101° C. |
| Kollidon SR | Polyvinyl acetate/polyvinylpyrrolidone |
| PEO_{100K} | polyethylene glycol; MW (ave) 100,000 |
| PPG | polypropylene glycol |
| PDLG | copolymer of DL-lactide and glycolide); inherent viscosity 1.6- |
| | 2.4 dl/g (CHCl₃) |
| Corbion PC17 | PURASORB ® Polycaprolactone; GMP grade homopolymer of ε- |
| | Caprolactone with an inherent viscosity midpoint of 1.7 dl/g |
| Corbion PC04 | PURASORB ® Polycaprolactone; GMP grade homopolymer of ε- |
| | Caprolactone with an inherent viscosity midpoint of 0.4 dl/g |
| PG | propylene glycol |
| Corbion | PURASORB ® 50/50 DL-lactide/glycolide copolymer; GMP |
| 5004/PURASORB ® | grade copolymer of DL-lactide and Glycolide in a 50/50 molar |
| PDLG 5004 | ratio and with an inherent viscosity midpoint of 0.4 dl/g |
| Corbion 5004A/ | PURASORB ® 50/50 DL-lactide/glycolide copolymer; acid |
| PURASORB ® | terminated GMP grade copolymer of DL-lactide and Glycolide in |
| PDLG 5004A | a 50/50 molar ratio and with an inherent viscosity midpoint of 0.4 |
| | dl/g |
| HPMCAS | Synthetic polymer derived from cellulose |
| P407 | Poloxamer 407; PEG-PPG-PEG triblock co-polymer |
| E172 | Ferrosoferric Oxide |

PLURONIC® is a registered trademark of BASF Corporation for polyoxyalkylene ethers. In any formulation described herein using trade names, the trade name can be replaced by the generic name. For example, a formulation described as comprising 50% Corbion PC17 and 50% Corbion PC04 is understood to describe a formulation comprising 50% polycaprolactone of viscosity 1.7 dl/g and 50% polycaprolactone of viscosity 0.4 dl/g.

### Gastric Residence System Drug Delivery Mechanism

Gastric residence dosage forms can be designed to be administered to the stomach of a patient by swallowing, by feeding tube, by gastric tube, etc. In some embodiments, the gastric residence dosage forms are folded into a compacted configuration and secured in a capsule. Once a gastric residence dosage form is in place in the stomach, it can open from its compacted form to an uncompacted form and remain in the stomach for a desired residence time (e.g., three days, seven days, two weeks, etc.). A gastric residence dosage form that is properly in place in a stomach will resist passage through the pyloric valve, which separates the stomach from the small intestine. Gastric residence dosage forms can release a therapeutic agent (i.e., API or drug) over the period of residence with controlled release. While residing in the stomach, the dosage form may not interfere with the normal passage of food or other gastric contents. Once the desired residence time has expired, the dosage form passes out of the stomach (i.e., through the pyloric valve) and is readily eliminated from the patient.

To administer a gastric residence system to a patient, the gastric residence system can be folded into a form small enough to be swallowed or otherwise administered. In some embodiments, the folded gastric residence system is retained in a capsule or other container which can be swallowed by the patient. In some cases, the gastric residence system may be delivered to a patient via gastrostomy tube, feeding tube, gastric tube, or other route of administration to the stomach. Specific examples of gastric residence systems may be found in PCT/US2018/051816, WO 2015/191920, WO 2017/070612, WO 2017/100367, WO 2018/064630, WO 2017/205844, WO 2018/227147, each of which is incorporated herein in its entirety.

Once the gastric residence system reaches the stomach of a patient, it may assume an open configuration. The dimensions of an open gastric residence system are, when left unaltered, suitable to prevent passage of the device through the pyloric valve for the period of time during which the device is intended to reside in the stomach. In some embodiments, the folded gastric residence system can also be secured by a dissolvable retaining band or sleeve that can prevent premature deployment of the gastric residence system in case of a failure of the capsule.

While in the stomach, the gastric residence system is compatible with digestion and other normal functioning of the stomach or gastrointestinal tract. The gastric residence system does not interfere with or impede the passage of chyme (partially digested food) or other gastric contents which exit the stomach through the pyloric valve into the duodenum.

Once released from the capsule into the stomach, the therapeutic agent (e.g., methadone) of the gastric residence system begins to take effect. In some embodiments, the gastric residence system comprises a plurality of carrier polymer-agent components. The carrier polymer-agent components may comprise a carrier polymer, a pore former, and a therapeutic agent (or a salt thereof). The plurality of carrier polymer-agent components are linked together by one or more coupling polymer components. The therapeutic agent may be eluted from the carrier polymer-agent components into the gastric fluid of the patient over the desired residence time of the system. Release of the therapeutic agent is controlled by appropriate formulation of the carrier polymer-agent components, including by the use of the dispersant in formulation of the carrier polymer-agent components, and by milling of the therapeutic agent to particles of desired size prior to blending the agent with the carrier polymer and dispersant.

Additionally, coatings can be applied to outer surfaces of the gastric residence system. The coatings can include additional therapeutic agents or agents that can affect the release of therapeutic agents or the residence duration of the gastric residence system.

Once the desired residence time has expired, the gastric residence system passes out of the stomach. To do so, various components of the gastric delivery system are designed to weaken and degrade. The specific dimensions of the system are also taken into consideration. In its intact, uncompacted, open configuration, the gastric residence system is designed to resist passage through the pyloric valve. However, coupling polymer components of the gastric residence system are chosen such that they gradually degrade over the specified residence period in the stomach. When the coupling polymer components are sufficiently weakened by degradation, the gastric residence system loses critical resilience to compression or size reduction and can break apart into smaller pieces. The reduced-size dosage form and any smaller pieces are designed to pass through the pyloric valve. The system then passes through the intestines and is eliminated from the patient. In some embodiments, a gastric residence system may be designed to weaken at specific locations such that the gastric residence system can pass through a pyloric valve intact once the residence time expires without degrading into numerous smaller pieces.

### Configuration & Components of Gastric Residence System

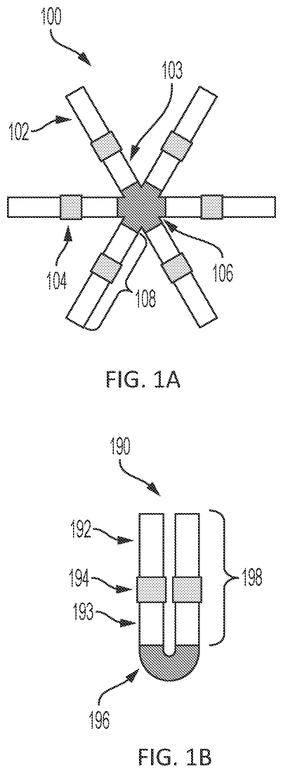

Gastric residence systems can be prepared in different configurations. The “stellate” configuration of a gastric residence system is also known as a “star” (or “asterisk”) configuration. An example of a stellate system 100 is shown schematically in FIG. 1A. Multiple arms (only one such arm, 108, is labeled for clarity), are affixed to disk-shaped central elastomer 106. The arms depicted in FIG. 1A are comprised of segments 102 and 103, joined by a coupling polymer or linker region 104 (again, the components are only labeled in one arm for clarity) which serves as a linker region. This configuration permits the system to be folded or compacted at the central elastomer. FIG. 1B shows a folded configuration 190 of the gastric residence system of FIG. 1A (for clarity, only two arms are illustrated in FIG. 1B). Segments 192 and 193, linker region 194, elastomer 196, and arm 198 of FIG. 1B correspond to segments 102 and 103, linker region 104, elastomer 106, and arm 108 of FIG. 1A, respectively. When folded, the overall length of the system is reduced by approximately a factor of two, and the system can be conveniently placed in a container such as a capsule or other container suitable for oral administration. The gastric residence system is constrained by the capsule or other container into the compacted state (the folded state). When the capsule reaches the stomach, the capsule dissolves, releasing the gastric residence system. Upon release of the constraint by the capsule or other container, the gastric residence system then unfolds into its uncompacted state, which is retained in the stomach for the desired residence period.

While the linker regions 104 are shown as slightly larger in diameter than the segments 102 and 103 in FIG. 1A, they can be the same diameter as the segments, so that the entire arm 102-104-103 has a smooth outer surface.

In some embodiments, the stellate system may have an arm composed of only one segment, which is attached to the central elastomer by a linker region. This corresponds to FIG. 1A with the segments 103 omitted. The single-segment arms comprising segments 102 are then directly attached to central elastomer 106 via the linkers 104. The linkers can comprise a coupling polymer or a disintegrating matrix.

A stellate system can be described as a gastric residence system for administration to the stomach of a patient, comprising an elastomer component, and at least one carrier polymer-agent component comprising a carrier polymer and an agent or a salt thereof, attached to the elastomer component, wherein each of the plurality of carrier polymer-agent components is an arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein each arm independently comprises one or more segments, each segment comprising a proximal end, a distal end, and an outer surface therebetween. In some embodiments, when two or more segments are present in an arm, each segment is attached to an adjacent segment via a linker region. In some embodiments, when two or more segments are present in an arm, one segment is directly attached to the other segment, without using a linker region. The linker region can be a coupling polymer or a disintegrating matrix. The arms can be attached to the central elastomer via a coupling polymer or a disintegrating matrix, and can have intervening portions of interfacing polymers. For the plurality of at least three arms, or for a plurality of arms, a preferred number of arms is six, but three, four, five, seven, eight, nine, or ten arms can be used. The arms should be equally spaced around the central elastomer; if there are N arms, there will be an angle of about 360/N degrees between neighboring arms.

### Coupling Polymers/Linker Regions

The coupling polymers of the gastric residence system, which serve as linker regions, are designed to break down gradually in a controlled manner during the residence period of the system in the stomach. If the gastric residence system passes prematurely into the small intestine in an intact form, the system is designed to break down much more rapidly to avoid intestinal obstruction. This is readily accomplished by using enteric polymers as coupling polymers. Enteric polymers are relatively resistant to the acidic pH levels encountered in the stomach, but dissolve at the higher pH levels found in the duodenum. Use of enteric coupling polymers as safety elements protects against undesired passage of the intact gastric residence system into the small intestine. In the system shown in FIG. 1A, at least the coupling polymer used for the couplings 104 are made from such enteric polymers.

In additional embodiments, a time-dependent coupling polymer or linker can be used. Such a time-dependent coupling polymer or linker degrades in a predictable, time-dependent manner. In some embodiments, the degradation of the time-dependent coupling polymer or linker may not be affected by the varying pH of the gastrointestinal system.

In additional embodiments, different types of linkers can be used in the gastric residence systems. That is, both enteric linkers (or enteric coupling polymers) and time-dependent linkers (or time-dependent coupling polymers) can be used. In some embodiments, a single multi-segment arm of a stellate system can use both an enteric linker at some linker regions between segments, and a time-dependent linker at other linker regions between segments.

Linker regions are typically about 100 microns to about 2 millimeter in width, such as about 200 um to about 2000 um, about 300 um to about 2000 um, about 400 um to about 2000 um, about 500 um to about 2000 um, about 600 um to about 2000 um, about 700 um to about 2000 um, about 800 um to about 2000 um, about 900 um to about 2000 um, about 1000 um to about 2000 um, about 1100 um to about 2000 um, about 1200 um to about 2000 um, about 1300 um to about 2000 um, about 1400 um to about 2000 um, about 1500 um to about 2000 um, about 1600 um to about 2000 um, about 1700 um to about 2000 um, about 1800 um to about 2000 um, or about 1900 um to about 2000 um; or about 100 um to about 1900 um, about 100 um to about 1800 um, about 100 um to about 1700 um, about 100 um to about 1600 um, about 100 um to about 1500 um, about 100 um to about 1400 um, about 100 to about 1300 um, about 100 um to about 1200 um, about 100 um to about 1100 um, about 100 um to about 1000 um, about 100 um to about 900 um, about 100 um to about 800 um, about 100 um to about 700 um, about 100 um to about 600 um, about 100 um to about 500 um, about 100 um to about 400 um, about 100 um to about 300 um, or about 100 um to about 200 um. Linker regions can be about 100 um, about 200 um, about 300 um, about 400 um, about 500 um, about 600 um, about 700 um, about 800 um, about 900 um, about 1000 um, about 1100 um, about 1200 um, about 1300 um, about 1400 um, about 1500 um, about 1600 um, about 1700 um, about 1800 um, about 1900 um, or about 2000 um in width, where each value can be plus or minus 50 um (±50 um).

### Central Elastomer

The central elastomeric polymer of a stellate system is typically not an enteric polymer; however, the central elastomeric polymer can also be made from such an enteric polymer where desirable and practical.

The central elastomer should have a specific durometer and compression set. The durometer is important because it determines the folding force of the dosage form and whether it will remain in the stomach; a preferred range is from about 60 to about 90 A. The compression set should be as low as possible to avoid having permanent deformation of the gastric residence system when stored in the capsule in its compacted configuration. A preferred range is about 10% to about 20% range. Liquid silicone rubber is a useful material for the central elastomer. Examples of materials that fit these requirements are the QP1 range of liquid silicone rubbers from Dow Corning. In any embodiment with a central elastomer, the QP1-270 (70 A durometer) liquid silicone rubber can be used. In some embodiments, the central elastomer may comprise a 50 A or 60 A durometer liquid silicone rubber (Shin Etsu).

### Segments/Arms

Segments and arms of the gastric residence systems can have cross-sections in the shape of a circle (in which case the segments are cylindrical), a polygon (such as segments with a triangular cross-section, rectangular cross-section, or square cross-section), or a pie-shaped cross-section (in which case the segments are cylindrical sections). Segments with polygon-shaped or pie-shaped cross-sections, and ends of cylindrically-shaped sections which will come into contact with gastric tissue, can have their sharp edges rounded off to provide rounded corners and edges, for enhanced safety in vivo. That is, instead of having a sharp transition between intersecting edges or planes, an arc is used to transition from one edge or plane to another edge or plane. Thus, a “triangular cross-section” includes cross-sections with an approximately triangular shape, such as a triangle with rounded corners. An arm with a triangular cross-section includes an arm where the edges are rounded, and the corners at the end of the arm are rounded. Rounded corners and edges are also referred to as fillet corners, filleted corners, fillet edges, or filleted edges.

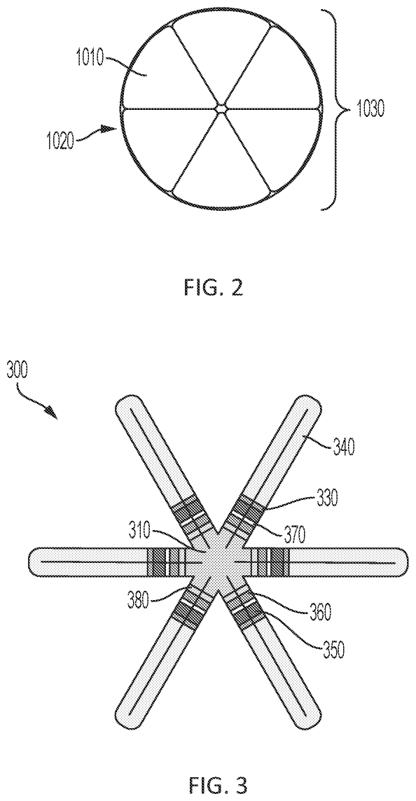

In some embodiments, the cross-section of the elongate members, or arms, used in a stellate gastric delivery system is that of a circular section, where the circular section is formed by two radii of the cylinder lying in the same plane and the arc that the radii intersect. The angle between the two radii (the central angle of the arc) is preferably about 360 degrees divided by 4, 6, or 8, but can be about 360 degrees divided by any integer between 2 and 12 inclusive. That is, a cross-section described as a circular section resembles a slice of pie, such as the cross-section depicted at the left of FIG. 2, and can be referred to as pie-shaped. Such a cross-section for the elongate member in a stellate system permits the gastric residence system to have an approximately cylindrical shape when compacted, as depicted at the right of FIG. 2 for a gastric residence system 1030 having six elongate members with wedge-shaped cross-sections (one elongate member, 1010, is labeled). The arrangement in FIG. 2 alleviates the stress on the containing capsule 1020 when the system is in its compacted form, as compared to an arrangement having elongate members with a triangular-shaped cross-section, and also permits more mass to be used in the elongate members, as less space in the capsule is wasted. Elongate members with such a cross-section can be produced via extrusion through a die having such a cross-section. For co-extrusion of multiple regions in a bulk configuration, such as an extruded slab or ribbon, compression molding or thermoforming can be used to form elongate members with such a cross-section from portions of the extruded bulk configuration.

In some embodiments, the stellate system is about 30 mm to about 60 mm when unfolded (arm extended). In some embodiments, the stellate system is about 41 mm to about 51 mm when unfolded. In some embodiments, the stellate system is about 45 mm to about 47 mm when unfolded. In some embodiments, the stellate system is about 46 mm when unfolded.

### Features for Improved Retention and Agent Release for Methadone Gastric Residence Systems

Retention of gastric residence systems for the desired residence period and agent release from gastric residence systems can be improved and made more consistent using the features described herein, such as a filament which is wrapped circumferentially around a gastric residence system and connecting the arms of the gastric residence system; use of timed linkers and enteric linkers which permit higher precision in retention and passage of the gastric residence system; and arms coated with release rate-modulating polymer films.

### Circumferential Filament

In some embodiments, a gastric residence system may comprise a circumferential filament. Gastric residence systems having a filament may help improve the gastric residence of the gastric residence system. Specifically, a filament can help provide a more consistent gastric residence time and/or a longer gastric residence time. Thus, gastric residence systems that include a filament may provide more predictable and/or controllable gastric residence times. Gastric residence systems having predictable and/or controllable gastric residence times can minimize the risk of the gastric residence system unfolding too early (e.g., in the esophagus) and causing an obstruction. Gastric residence systems having predictable and/or controllable gastric residence times can also minimize the possibility of the gastric residence system passing through the stomach and unfolding later in the gastrointestinal tract (i.e., intestine), or passing through the gastrointestinal tract without unfolding at all. In each of these possible scenarios, the therapeutic agent of the gastric residence dosage form is not delivered to the patient as intended.

However, it has been demonstrated that gastric residence systems of a stellate shape can bend into a configuration that allows for premature passage through the pylorus of a patient. Gastric residence systems that prematurely pass through the pylorus fail to deliver the therapeutic agent of the gastric residence system to the patient. Further, premature passage causes inconsistency, causes unreliability, and compromises the efficacy of the gastric residence system.

The feature of circumferential filament is described in International Patent Application PCT/US2020/059541, which is hereby incorporated by reference in its entirety.

### Time-Dependent Linkers (Timed Disintegrating Matrices) and Enteric Linkers (Enteric Disintegrating Matrices)

Described below are polymeric linkers (e.g., timed linkers and/or enteric linkers) and times-dependent linkers specifically.

### Polymeric Linkers

The agent-containing structural members are attached to a second structural member (such as a central member, which may be an elastic central member) through one or more linkers. A polymeric linker may directly interface with the agent-containing structural member, or may interface with the agent-containing structural member through a coupling member. Similarly, the polymeric linker may interface directly with the second structural member, or may interface through a coupling member. In an embodiment wherein the agent-containing structural member is connected to the second structural member through two or more polymeric linkers, the polymeric linkers may directly interface with each other, or may interface through a coupling member. One or both of an enteric linker and a time-dependent linkers may be used, or a polymeric linker may function as both an enteric linker and a time-dependent linker.

The polymeric linkers are typically about 100 microns to about 3 millimeter in width, such as about 200 um to about 3000 um, about 300 um to about 3000 um, about 400 um to about 3000 um, about 500 um to about 3000 um, about 600 um to about 3000 um, about 700 um to about 3000 um, about 800 um to about 3000 um, about 900 um to about 3000 um, about 1000 um to about 3000 um, about 1100 um to about 3000 um, about 1200 um to about 3000 um, about 1300 um to about 3000 um, about 1400 um to about 3000 um, about 1500 um to about 3000 um, about 1600 um to about 3000 um, about 1700 um to about 3000 um, about 1800 um to about 3000 um, about 1900 um to about 3000 um, about 2000 um to about 3000 um, about 2100 um to about 3000 um, about 2200 um to about 3000 um, about 2300 um to about 3000 um, about 2400 um to about 3000 um, about 2500 um to about 3000 um, about 2600 um to about 3000 um, about 2700 um to about 3000 um, about 2800 um to about 3000 um, or about 2900 um to about 3000 um; or about 100 um to about 200 um, about 200 um to about 300 um, about 300 um to about 400 um, about 400 um to about 500 um, about 500 um to about 600 um, about 600 um to about 700 um, about 700 um to about 800 um, about 800 um to about 900 um, about 900 um to about 1000 um, about 1000 um to about 1100 um, about 1100 um to about 1200 um, about 1200 um to about 1300 um, about 1300 um to about 1400 um, about 1400 um to about 1500 um, about 1500 um to about 1600 um, about 1600 um to about 1700 um, about 1700 um to about 1800 um, about 1800 um to about 1900 um, about 1900 um to about 2000 um, about 2000 um to about 2100 um, about 2100 um to about 2200 um, about 2200 um to about 2300 um, about 2300 um to about 2400 um, about 2400 um to about 2500 um, about 2500 um to about 2600 um, about 2600 um to about 2700 um, about 2700 um to about 2800 um, about 2800 um to about 2900 um, about 2900 um to about 3000 um. Polymeric linkers can be about 100 um, about 200 um, about 300 um, about 400 um, about 500 um, about 600 um, about 700 um, about 800 um, about 900 um, about 1000 um, about 1100 um, about 1200 um, about 1300 um, about 1400 um, about 1500 um, about 1600 um, about 1700 um, about 1800 um, about 1900 um, about 2000 um, about 2100 um, about 2200 um, about 2300 um, about 2400 um, about 2500 um, about 2600 um, about 2700 um, about 2800 um, about 2900 um, about 3000 um in width, where each value can be plus or minus 50 um (±50 um).

The cross section of the polymeric linker may be round (i.e., circular), elliptical, triangular, square, rectangular, pentagonal, hexagonal, pie-shaped or any other polymeric shape. In some embodiments, the cross-section of the polymeric linker is the same shape as the cross-section of an agent-containing structural member attached to the polymeric linker. In some embodiments, the cross-section of the polymeric linker has a larger area than the cross-section of the agent-containing structural member, a smaller area than the cross-section of the agent-containing structural member, or approximately the same area as the cross-section of the attached agent-containing structural member.

In some embodiments, a polymeric linker may comprise a polylactic acid (PLA), a polycaprolactone (PCL), or another suitable polymer.

### Time-Dependent Disintegrating Matrices (Time-Dependent Linkers)

A time-dependent linker degrades in a predictable, time-dependent manner under aqueous conditions, such as when the gastric residence system is deployed in the stomach of an individual. The time-dependent polymeric linkers control the residence time of the gastric residence system in the stomach. The time-dependent polymeric linkers are designed to degrade, dissolve, mechanically weaken, or break gradually over time. After the desired residence period, the time-dependent polymeric linker has degraded, dissolved, disassociated, or mechanically weakened, or has broken, to the point where the gastric residence system can pass through the pyloric valve, exiting the gastric environment and entering the small intestine, for eventual elimination from the body.

The time-dependent polymeric linker preferably comprises a pH-independent degradable polymer, which degrades under aqueous conditions in a pH-independent or approximately pH-independent manner. Exemplary pH-independent degradable polymer include PLGA, PLA, PCL, polydioxanone, cellulose, or blends or copolymers thereof.

The time-dependent polymeric linker can include poly (lactic-co-glycolide) (PLGA).

In some embodiments, the PLGA of the time-dependent polymeric linker comprises copolymer of DL-lactide and glycolide (50/50 molar ratio) having a viscosity midpoint between about 0.32 dl/g to about 0.48 dl/g (such as about 0.4 dl/g) (such as the PLGA sold under the tradename Purasorb® PDLG 5004, available from Corbion). In some embodiments, the PLGA of the time-dependent polymeric linker comprises acid terminated copolymer of DL-lactide and glycolide (50/50 molar ratio) having a viscosity midpoint between about 0.32 dl/g to about 0.48 dl/g (such as about 0.4 dl/g) (such as the PLGA sold under the tradename Purasorb® PDLG 5004A available from Corbion). In some embodiments, the PLGA of the time-dependent polymeric linker comprises a mixture of (a) poly (D,L-lactic-co-glycolide) with a ratio of lactide monomers to glycolide monomers of about 50:50 (such as the PLGA sold under the tradename Purasorb® PDLG 5004, available from Corbion), and (b) acid-terminated poly (D,L-lactic-co-glycolide) with a ratio of lactide monomers to glycolide monomers of about 50:50 (such as the PLGA sold under the tradename Purasorb® PDLG 5004A, available from Corbion).

The one or more additional linker polymers included in the polymer linker may be homogenously mixed with the PLGA. In some embodiments, the one or more additional linker polymers are miscible with the PLGA. The one or more additional linker polymers may be a non-degradable polymer (that is, not degradable or in the gastric or enteric environment, or an aqueous solution of pH 1.6 (representing the gastric environment) or pH 6.5 (representing the enteric environment), and is optionally present in the time-dependent polymeric linker is an amount such that the time-dependent polymeric linker does not break during the gastric residence period.

Bonding of the polymeric linker to a directly adjacent member may be improved if at least one polymer is common to both the adjacent member and the time-dependent polymeric linker. In some embodiments, the at least one common polymer is polycaprolactone (PCL).

In some embodiments, the one or more additional linker polymers comprises a PCL. The time-dependent polymeric linker may be directly joined or bonded to another member of the gastric residence system (such as the structural member comprising the drug and the carrier polymer, a coupling member, the enteric polymeric linker, or a central structural member), which may also include a PCL, which may be the same PCL in the time-dependent polymeric linker or a different PCL as the one in the polymeric linker, and which may be at the same concentration or a different concentration. A different PCL in the time-dependent polymeric linker and the other member directly joined or bonded to the time-dependent linker may differ, for example, in the weight-average molecular weight of the PCL, the inherent viscosity of the PCL, or the proportions of PCL (for example, when a blend of two or more PCL polymers are used). In some embodiments, the time-dependent disintegrating matrix comprises about 40 wt % to about 50 wt % PCL. In some embodiments, the time-dependent disintegrating matrix comprises about 43 wt % to about 47 wt % PCL. In some embodiments, the time-dependent disintegrating matrix comprises about 45 wt % PCL. In some embodiments, the time-dependent disintegrating matrix comprises about 44.95 wt % PCL.

The time-dependent polymeric linker may further include one or more plasticizers, such as polyethylene glycol. The term “polyethylene glycol” is used interchangeably herein with the terms “polyethylene oxide” and “PEO.” In some embodiments, the molecular weight of the polyethylene glycol is about 90K to about 110K, such as 100 k (also referred to as 100K or 100 kDa. In some embodiments, the time-dependent disintegrating matrix comprises polyethylene glycol with molecular weight of about 100 k (polyethylene glycol 100 k). In some embodiments, the time-dependent disintegrating matrix comprises about 0.5 wt % to about 5 wt % polyethylene glycol 100 k. In some embodiments, the time-dependent disintegrating matrix comprises about 1 wt % to about 3 wt % polyethylene glycol 100 k. In some embodiments, the time-dependent disintegrating matrix comprises about 2 wt % polyethylene glycol 100 k.

In some embodiments, the time-dependent disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The time-dependent disintegrating matrix may include the color-absorbing dye in an amount of up to about 5%, such as up to about 4%, up to about 3%, up to about 2%, up to about 1%, up to about 0.5%, up to about 0.3%, up to about 0.2%, up to about 0.1%, or up to about 0.05%. In some embodiments, the time-dependent disintegrating matrix comprises about 0.005 wt % to about 0.2 wt % color-absorbing dye. In some embodiments, the time-dependent disintegrating matrix comprises about 0.01 wt % to about 0.1 wt % color-absorbing dye. In some embodiments, the time-dependent disintegrating matrix comprises about 0.05 wt % color-absorbing dye. In some embodiments, the color-absorbing dye is E172.

In one example of a time-dependent disintegrating matrix, the time-dependent disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172.

In another example of a time-dependent disintegrating matrix, the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172.

In another example of a time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172.

In some embodiments, a dosage form for administration of one or more agents comprises a gastric residence system, wherein the gastric residence system comprises a time-dependent disintegrating matrix comprising about 44.95 wt % of polycaprolactone (PCL), such as PCL having a viscosity midpoint between about 1.5 dl/g to about 2.1 dl/g, such as Corbion PC17. In some embodiments, the gastric residence system comprises a time-dependent disintegrating matrix comprising about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the gastric residence system comprises a time-dependent disintegrating matrix comprising about 18.0 wt % of a copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the gastric residence system comprises a time-dependent disintegrating matrix comprising about 2.0 wt % of polyethylene glycol, such as polyethylene glycol with average molecular weight of 100,000, such as PEO100K. In some embodiments, the gastric residence system comprises a time-dependent disintegrating matrix comprising about 0.05 wt % of iron oxide, such as E172. In some embodiments, a dosage form for administration of one or more agents comprises a gastric residence system, wherein the gastric residence system comprises a time-dependent disintegrating matrix comprising about 44.95 wt % of Corbion PC17, about 35.0 wt % of PDLG 5004A, about 18.0 wt % of PDLG 5004, about 2.0 wt % of PEO100K, and about 0.05 wt % of E172.

Exemplary amounts of the components for the time-dependent disintegrating matrix are provided in the table below. The amounts are given in approximate weight percent, with the understanding that when ranges are provided, the amounts are chosen so as to add up to 100%.

| Time-dependent | | | |
|---|---|---|---|
| disintegrating matrix | Formulation 1 | Formulation 2 | Formulation 3 |
| PCL | 40-50 | 43-47 | 44.95 |
| PDLG5004A | 30-40 | 33-37 | 35 |
| PDLG5004 | 10-25 | 15-20 | 18 |
| PEO(100k) | 0.5-5 | 1-3 | 2 |
| coloring (optional) | 0.005-0.2 | 0.01-0.1 | 0.05 (e.g., E172) |

### Enteric Disintegrating Matrices (Enteric Linkers)

The pH-dependent disintegrating matrices provide a safety mechanism for the gastric residence systems. If the system exits the stomach prematurely, that is, with all of the time-dependent disintegrating matrices intact, the pH-dependent disintegrating matrices will degrade, dissolve, disassociate, or mechanically weaken in the high pH environment of the small intestine, permitting the gastric residence system to pass readily through the small intestine. In addition, after passage of the gastric residence system once the time-dependent disintegrating matrices degrade, dissolve, disassociate, or mechanically weaken in the gastric environment, exposure of the pH-dependent disintegrating matrices to the high pH of the small intestine will provide further weakening and/or break-up of the system, for ready passage through the small intestine.

If the gastric residence system passes prematurely into the small intestine in an intact form, the system may be designed to break down much more rapidly to avoid intestinal obstruction. This is readily accomplished by using an enteric polymeric linker that includes an enteric polymer in addition to an additional linker polymer (such as a carrier polymer), which weakens or degrades within the intestinal environment. Enteric polymers are relatively resistant to the acidic pH levels encountered in the stomach, but dissolve rapidly at the higher PH levels found in the duodenum. Use of enteric polymeric linkers as safety elements protects against undesired passage of the intact gastric residence system into the small intestine. The use of enteric polymeric linker also provides a manner of removing the gastric residence system prior to its designed residence time; should the system need to be removed, the patient can drink a mildly alkaline solution, such as a sodium bicarbonate solution, or take an antacid preparation such as hydrated magnesium hydroxide (milk of magnesia) or calcium carbonate, which will raise the pH level in the stomach and cause rapid degradation of the enteric polymeric linker.

Weakening or degradation of the enteric polymeric linker may be measured in references to a loss of the flexural modulus or breakage of the polymeric linker under a given condition (e.g., enteric conditions or gastric conditions). The enteric linkers weaken, degrade, or break in the intestinal environment relatively quickly, while retain much of their flexural modulus in the gastric environment. Stomach conditions may be simulated using an aqueous solution, such FaSSGF, at a pH of 1.6 and at 37° C., and intestinal conditions may be simulated using an aqueous solution, such as FaSSIF, at a pH 6.5 at 37° C.

In some embodiments, the enteric disintegrating matrix comprises hydroxypropyl methylcellulose acetate succinate (HPMCAS). For example, in some embodiments, the enteric disintegrating matrix includes about 60 wt % to about 70 wt % HPMCAS. In some embodiments, the enteric disintegrating matrix includes about 62 wt % to about 66 wt % HPMCAS. In some embodiments, the enteric disintegrating matrix includes about 63.95 wt % HPMCAS.

The enteric polymer is combined with one or more additional polymers (such as one or more carrier polymers) in the enteric linker, preferably in a homogenous mixture. For example, the enteric polymer and the additional linker polymer may be homogenously blended together before the mixture is extruded, and the extruded material being cut to a desired size for the polymeric linker. In some embodiments, the one or more additional linker polymers are miscible with the enteric polymer. The one or more additional linker polymers may be a non-degradable polymer (that is, not degradable or in the gastric or enteric environment, or an aqueous solution of pH 1.6 (representing the gastric environment) or pH 6.5 (representing the enteric environment).

Bonding of the polymeric linker to a directly adjacent member may be improved if at least one polymer is common to both the adjacent member and the enteric polymeric linker. That is, one of the one or more additional linker polymers in the enteric linker may be the same (or the same polymer type) as at least one polymer in a directly adjacent component (or, optionally, both directly adjacent components) of the gastric residence system. For example, if the enteric polymeric linker is bonded directly to a structural member comprising a carrier polymer, in some embodiments the one or more additional linker polymers also includes the carrier polymer (in addition to the PLGA in the time-dependent polymeric linker) at the same or different concentration. Exemplary carrier polymers include, but are not limited to, polylactic acid (PLA), and polycaprolactone (PCL), among others described herein.

In some embodiments, the one or more additional linker polymers in the enteric linker comprises a PCL. The enteric polymeric linker may be directly joined or bonded to another member of the gastric residence system (such as the structural member comprising the drug and the carrier polymer, a coupling member, the time-dependent polymeric linker, or a central structural member), which may also include a PCL, which may be the same PCL in the enteric polymeric linker or a different PCL as the one in the enteric polymeric linker, and which may be at the same concentration or a different concentration. A different PCL in the enteric polymeric linker and the other member directly joined or bonded to the enteric linker may differ, for example, in the weight-average molecular weight of the PCL, the inherent viscosity of the PCL, or the proportions of PCL (for example, when a blend of two or more PCL polymers are used). In some embodiments, the enteric disintegrating matrix comprises about 30 wt % to about 40 wt % PCL. In some embodiments, the enteric disintegrating matrix comprises about 32 wt % to about 37 wt % PCL. In some embodiments, the enteric disintegrating matrix comprises about 34 wt % PCL. In some embodiments, the enteric disintegrating matrix comprises about 33.95 wt % PCL.

The enteric disintegrating matrix may further include one or more plasticizers, such as a poloxamer (e.g., Poloxamer 407, or “P407”). In some embodiments, the enteric disintegrating matrix comprises about 0.5 wt % to about 5 wt % poloxamer. In some embodiments, the enteric disintegrating matrix comprises about 1 wt % to about 3 wt % poloxamer. In some embodiments, the enteric disintegrating matrix comprises about 2 wt % poloxamer.

In some embodiments, the enteric disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The enteric polymeric linker may include the color-absorbing dye in an amount of up to about 5%, such as up to about 4%, up to about 3%, up to about 2%, up to about 1%, up to about 0.5%, up to about 0.3%, up to about 0.2%, or up to about 0.1%. In some embodiments, the enteric disintegrating matrix comprises about 0.01 wt % to about 0.2 wt % color-absorbing dye ferrosoferric oxide. In some embodiments, the enteric disintegrating matrix comprises about 0.05 wt % to about 0.15 wt % color-absorbing dye ferrosoferric oxide. In some embodiments, the enteric disintegrating matrix comprises about 0.1 wt % color-absorbing dye ferrosoferric oxide.

In some embodiments, the enteric disintegrating matrix comprises about 59 wt % to about 69 wt % HPMCAS, about 29 wt % to about 39 wt % PCL, and about 0.5 wt % to about 5 wt % poloxamer (such as P407). Optionally, the enteric disintegrating matrix further comprises iron oxide, for example about 0.01 wt % to about 0.2 wt % iron oxide (such as E172).

In some embodiments, the enteric disintegrating matrix comprises about 62 wt % to about 66 wt % HPMCAS, about 32 wt % to about 36 wt % PCL, and about 1 wt % to about 3 wt % poloxamer (such as P407). Optionally, the enteric disintegrating matrix further comprises iron oxide, for example about 0.05 wt % to about 0.15 wt % iron oxide (such as E172).

In some embodiments, the enteric disintegrating matrix comprises about 63.95 wt % HPMCAS, about 33.95 wt % PCL, and about 2 wt % poloxamer (such as P407). Optionally, the enteric disintegrating matrix further comprises iron oxide, for example about 0.1 wt % iron oxide (such as E172).

In some embodiments, a dosage form for administration of one or more agents comprises a gastric residence system, wherein the gastric residence system comprises a pH-dependent disintegrating matrix comprising about 33.95 wt % of polycaprolactone (PCL), such as PCL having a viscosity midpoint between about 1.5 dl/g to about 2.1 dl/g, such as Corbion PC17. In some embodiments, the gastric residence system comprises a pH-dependent disintegrating matrix comprising about 63.95 wt % of hypromellose acetate succinate, such as HPMCAS-MG. In some embodiments, the gastric residence system comprises a pH-dependent disintegrating matrix comprising about 2.0 wt % of poly (ethylene glycol)-block-poly (propylene glycol)-block-poly (ethylene glycol) polymers, such as H—(OCH2CH2)x—(O—CH(CH3)CH2)y—(OCH2CH2)z—OH where x and z are about 101 and y is about 56, such as Poloxamer 407 (P407, a poly (ethylene glycol)-block-poly (propylene glycol)-block-poly (ethylene glycol) polymer with a polyoxypropylene molecular mass of about 4000 and about 70% polyoxyethylene content). In some embodiments, the gastric residence system comprises a pH-dependent disintegrating matrix comprising about 0.1 wt % of iron oxide, such as E172. In some embodiments, a dosage form for administration of one or more agents comprises a gastric residence system, wherein the gastric residence system comprises a pH-dependent disintegrating matrix comprising about 33.95 wt % of Corbion PC17, about 63.95 wt % of HPMCAS-MG, about 2.0 wt % of P407, and about 0.1 wt % of E172.

Exemplary amounts of the components for the enteric disintegrating matrix are provided in the table below. The amounts are given in approximate weight percent, with the understanding that when ranges are provided, the amounts are chosen so as to add up to 100%.

| Enteric | | | |
|---|---|---|---|
| disintegrating matrix | Formulation 1 | Formulation 2 | Formulation 3 |
| PCL | 29-39 | 32-36 | 33.95 |
| HPMCAS | 59-69 | 62-66 | 63.95 |
| P407 | 0.5-5 | 1-3 | 2 |
| coloring (optional) | 0.01-0.2 | 0.05-0.15 | 0.1 (e.g. E172) |

### Arm Tip Disintegrating Matrices

In some embodiments, the gastric residence system comprises arms comprising a third disintegrating matrix in addition to the time-dependent disintegrating matrix and the enteric disintegrating matrix. In some embodiments, the third disintegrating matrix is a filament holding segment (i.e., segment to which the filament is attached). In some embodiments, the third disintegrating is the distal segment of the residence system arm, i.e., the tip of the arm.

In some embodiments, the third disintegrating matrix comprises hydroxypropyl methylcellulose acetate succinate (HPMCAS). For example, in some embodiments the third disintegrating matrix includes about 60 wt % to about 70 wt % HPMCAS. In some embodiments, the third disintegrating matrix includes about 63 wt % to about 67 wt % HPMCAS. In some embodiments, the third disintegrating matrix includes about 64.9 wt % HPMCAS.

In some embodiments, the third disintegrating matrix comprises a polymer common with one or other segment in the gastric residence system arm. In some embodiments, the third disintegrating matrix comprises polycaprolactone (PCL). In some embodiments, the third disintegrating matrix comprises about 25 wt % to about 35 wt % PCL. In some embodiments, the third disintegrating matrix comprises about 28 wt % to about 32 wt % PCL. In some embodiments, the third disintegrating matrix comprises about 30 wt % PCL.

In some embodiments, the third disintegrating matrix comprises one or more acids, such as stearic acid. In some embodiments, the third disintegrating matrix comprises about 1 wt % to about 5 wt % stearic acid. In some embodiments, the third disintegrating matrix comprises about 2 wt % to about 3 wt % stearic acid. In some embodiments, the third disintegrating matrix comprises about 2.5 wt % stearic acid.

In some embodiments, the third disintegrating matrix may further include one or more plasticizers, such as a propylene glycol. In some embodiments, the third disintegrating matrix comprises about 1 wt % to about 5 wt % propylene glycol. In some embodiments, the third disintegrating matrix comprises about 2 wt % to about 3 wt % propylene glycol. In some embodiments, the third disintegrating matrix comprises about 2.5 wt % propylene glycol.

In some embodiments, the third disintegrating matrix includes a color-absorbing dyes (also referred to as a colorant or a pigment). A color-absorbing dye may be included to enhance bonding or attachment of the polymeric linker to other gastric residence system components. Color-absorbing dyes can absorb heat during the laser-welding, infrared welding, or other heat-induced attachment, which increases the tensile strength of the resulting bond. Exemplary color-absorbing dyes include iron oxide and carbon black. The third disintegrating matrix may include the color-absorbing dye in an amount of up to about 5%, such as up to about 4%, up to about 3%, up to about 2%, up to about 1%, up to about 0.5%, up to about 0.3%, up to about 0.2%, or up to about 0.1%. In some embodiments, the third disintegrating matrix comprises about 0.01 wt % to about 0.5 wt % color-absorbing dye. In some embodiments, the third disintegrating matrix comprises about 0.05 wt % to about 0.15 wt % color-absorbing dye. In some embodiments, the third disintegrating matrix comprises about 0.1 wt % color-absorbing dye. In some embodiments, the third disintegrating matrix comprises about 0.025% ferrosoferric oxide and about 0.075% FD&C Red 40. In some embodiments, the third disintegrating matrix comprises about 0.025% ferrosoferric oxide and about 0.075% FD&C Red 40.

In some embodiments, the third disintegrating matrix comprises about 60 wt % to about 70 wt % HPMCAS, about 25 wt % to about 35 wt % PCL, about 1 wt % to about 5 wt % propylene glycol and about 1 wt % to about 5 wt % stearic acid. Optionally, the third disintegrating matrix further comprises about 0.01 wt % to about 0.5 wt % iron oxide.

In some embodiments, the third disintegrating matrix comprises about 63 wt % to about 67 wt % HPMCAS, about 28 wt % to about 32 wt % PCL, about 2 wt % to about 3 wt % propylene glycol and about 2 wt % to about 3 wt % stearic acid. Optionally, the third disintegrating matrix further comprises about 0.05 wt % to about 0.15 wt % iron oxide.

In some embodiments, the third disintegrating matrix comprises 64.9 wt % HPMCAS, about 30 wt % PCL, about 2.5 wt % propylene glycol and about 2.5 wt % stearic acid. Optionally, the third disintegrating matrix further comprises about 0.1 wt % iron oxide, for example about 0.025% ferrosoferric oxide and about 0.075% FD&C Red 40.

Exemplary amounts of the components for the third disintegrating matrix are provided in the table below. The amounts are given in approximate weight percent, with the understanding that when ranges are provided, the amounts are chosen so as to add up to 100%.

| ODMTEP | | | |
|---|---|---|---|
| disintegrating | | | |
| matrix | Formulation 1 | Formulation 2 | Formulation 3 |
| PCL | 25-35 | 28-32 | 30 |
| HPMCAS | 60-70 | 63-67 | 64.9 |
| Stearic acid | 1-5 | 2-3 | 2.5 |
| Propylene Glycol | 1-5 | 2-3 | 2.5 |
| Iron oxide | 0.01-0.5 | 0.05-0.15 | 0.1 (e.g. 0.025% |
| | | | ferrosoferric oxide |
| | | | and 0.075% FD&C |
| | | | Red 40) |

### Inert Segments

In some embodiments, the gastric residence system comprises one or more inert segments. In some embodiments, the inert segment comprises one or more radiopaque substances.

In some embodiments, the inert segment comprises a common polymer with other segments in the gastric residence system. In some embodiments, the inert segment comprises polycaprolactone (PCL). In some embodiments, the inert segment comprises about 61 wt % to about 71 wt % PCL. In some embodiments, the inert segment comprises about 64 wt % to about 69 wt % PCL. In some embodiments, the inert segment comprises about 66.5 wt % PCL. In some embodiments, the inert segment comprises about 66.45 wt % PCL.

In some embodiments, the inert segment comprises vinylpyrrolidone-vinyl acetate copolymer in a ratio of 6:4 by mass (i.e. copovidone, such as Kollidon VA64). In some embodiments, the inert segment comprises about 27 wt % to about 37 wt % copovidone. In some embodiments, the inert segment comprises about 30 wt % to about 34 wt % copovidone. In some embodiments, the inert segment comprises about 32 wt % copovidone.

The inert segment may further include one or more plasticizers, such as a poloxamer (e.g., Poloxamer 407, or “P407”). In some embodiments, the inert segment comprises about 0.2 wt % to about 4 wt % poloxamer. In some embodiments, the inert segment comprises about 0.5 wt % to about 2.5 wt % poloxamer. In some embodiments, the inert segment comprises about 1.5 wt % poloxamer.

In some embodiments, the inert segment includes a color-absorbing dyes (also referred to as a colorant or a pigment). The inert segment may include the color-absorbing dye in an amount of up to about 5%, such as up to about 4%, up to about 3%, up to about 2%, up to about 1%, up to about 0.5%, up to about 0.3%, up to about 0.2%, up to about 0.1%, or up to 0.05%. In some embodiments, the inert segment comprises about 0.005 wt % to about 0.2 wt % color-absorbing dye. In some embodiments, the inert segment comprises about 0.01 wt % to about 0.1 wt % color-absorbing dye. In some embodiments, the inert segment comprises about 0.05 wt % color-absorbing dye. In some embodiments, the color-absorbing dye is FD&C Blue #1 Alum Lake.

In some embodiments, the inert segment comprises about 61 wt % to about 71 wt % PCL, about 27 wt % to about 37 wt % copovidone, about 0.2 wt % to about 4 wt % poloxamer. Optionally, the inert segment further comprises color-absorbing dye, for example about 0.005 wt % to about 0.2 wt % color-absorbing dye FD&C Blue #1 Alum Lake.

In some embodiments, the inert segment comprises about 64 wt % to about 69 wt % PCL, about 30 wt % to about 34 wt % copovidone, about 0.5 wt % to about 2.5 wt % poloxamer. Optionally, the inert segment further comprises color-absorbing dye, for example about 0.01 wt % to about 0.1 wt % color-absorbing dye FD&C Blue #1 Alum Lake.

In some embodiments, the inert segment comprises about 66.45 wt % PCL, about 32 wt % copovidone, about 1.5 wt % poloxamer. Optionally, the inert segment further comprises color-absorbing dye, for example about 0.05 wt % color-absorbing dye FD&C Blue #1 Alum Lake.

Exemplary amounts of the components for one embodiment of the inert segment (e.g. inactive spacer) are provided in the table below. In some embodiments herein, segments of the formulations provided in the table immediately below are referred to as “inactive” segments. An inactive segment may not include a radioactive substance. The amounts are given in approximate weight percent, with the understanding that when ranges are provided, the amounts are chosen so as to add up to 100%.

| Inert Segment | | | |
|---|---|---|---|
| (Inactive spacer) | Formulation 1 | Formulation 2 | Formulation 3 |
| PCL | 61-71 | 64-69 | 66.45 |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. |
| | | | Blue #1) |

In some embodiments, the gastric residence system comprises one or more inert segments, wherein the inert segment comprises one or more radiopaque substances. In some embodiments, the gastric residence system comprises an inert segment, wherein the inert segment is a radiopaque segment.

In some embodiments, the inert segment comprises a common polymer with other segments in the gastric residence system. In some embodiments, the inert segment comprises polycaprolactone (PCL). In some embodiments, the inert segment comprises about 65 wt % to about 75 wt % PCL. In some embodiments, the inert segment comprises about 68 wt % to about 72 wt % PCL. In some embodiments, the inert segment comprises about 70 wt % PCL.

In some embodiments, the inert segment comprises a radiopaque substance. In some embodiments, the inert segment comprises a radiopaque substance, wherein the radiopaque substance is (BiO)2CO3. In some embodiments, the inert segment comprises (BiO)2CO3. In some embodiments, the inert segment comprises about 25 wt % to about 35 wt % (BiO)2CO3. In some embodiments, the inert segment comprises about 28 wt % to about 32 wt % (BiO)2CO3. In some embodiments, the inert segment comprises about 30 wt % (BiO)2CO3.

In some embodiments, the inert segment comprises about 65 wt % to about 75 wt % PCL, and about 25 wt % to about 35 wt % (BiO)2CO3. In some embodiments, the inert segment comprises about 68 wt % to about 72 wt % PCL, and about 28 wt % to about 32 wt % (BiO)2CO3. In some embodiments, the inert segment comprises about 70 wt % PCL, and about 30 wt % (BiO)2CO3.

Exemplary amounts of the components for one embodiment of the inert segment (e.g. rPCL segment) are provided in the table below. The amounts are given in approximate weight percent, with the understanding that when ranges are provided, the amounts are chosen so as to add up to 100%.

| Inert segment- | | | |
|---|---|---|---|
| rPCL (radiopaque) | Formulation 1 | Formulation 2 | Formulation 3 |
| PCL | 65-75 | 68-72 | 70 |
| (BiO)₂CO₃ | 25-35 | 28-32 | 30 |

### Carrier Polymer-Agent Segments (Drug-Eluting Segments) Comprising a Methadone Formulation

The carrier polymer-agent segments, or drug-eluting segments, release an agent in a controlled manner during the period that the gastric residence system resides in the stomach. The carrier polymer may be blended with the agent, and formed into segments which are then assembled with the other components described herein to manufacture the gastric residence system. The composition of such carrier polymer-agent blends is provided below for specific drug formulations, including formulations comprising methadone.

In some embodiments, a dosage form for administration of methadone comprises a gastric residence system comprising about 10 mg to about 320 mg of methadone. In some embodiments, a dosage form for administration of methadone comprises a gastric residence system comprising about 50 mg to about 300 mg of methadone. In some embodiments, a dosage form for administration of methadone comprises a gastric residence system comprising less than or equal to about 320, about 300, about 280, about 260, about 240, about 220, about 200, about 180, about 160, about 140, about 120, about 100, about 80, about 60, about 40, or about 20 mg of methadone. In some embodiments, a dosage form for administration of methadone comprises a gastric residence system comprising greater than or equal to about 10, about 20, about 40, about 60, about 80, about 100, about 120, about 140, about 160, about 180, about 200, about 220, about 240, about 260, about 280, or about 300 mg methadone.

In some embodiments, the dosage form comprises a gastric residence system, wherein the gastric residence system comprises one or more drug-eluting segments, each of the one or more drug-eluting segments comprising about 10 mg to about 60 mg methadone. In some embodiments, each drug-eluting segment comprises less than or equal to about 60, about 50, about 40, about 30, or about 20 mg methadone. In some embodiments, each drug-eluting segment comprises more than or equal to about 10, about 20, about 30, about 40, or about 50 mg methadone.

In some embodiments, a methadone formulation comprises about 40 wt % to about 70 wt % methadone. In some embodiments, a methadone formulation comprises less than or equal to about 70, 65, 60, 55, 50, or 45 wt % methadone. In some embodiments, a methadone formulation comprises greater than or equal to about 40, 45, 50, 55, 60, or 65 wt % methadone.

Methadone formulations comprise about 30 wt % to 50 wt % polycaprolactone (PCL), such as PCL having a viscosity midpoint between about 1.5 dl/g to about 2.1 dl/g, such as Corbion PC17. In some embodiments, a methadone formulation comprises less than or equal to about 50, about 45, about 40, or about 35 wt % PCL. In some embodiments, a methadone formulation comprises greater than or equal to about 30, about 35, about 40, or about 45 wt % PCL.

In some embodiments, a methadone formulation comprises about 0.1 wt % to about 5 wt % of poly (ethylene glycol)-block-poly (propylene glycol)-block-poly (ethylene glycol) polymers, such as H—(OCH2CH2)x—(O—CH(CH3)CH2)y—(OCH2CH2)z—OH where x and z are about 101 and y is about 56, such as Poloxamer 407. In some embodiments, a methadone formulation comprises less than or equal to about 5, about 4, about 3, about 2, about 1, or about 0.5 wt % poly (ethylene glycol)-block-poly (propylene glycol)-block-poly (ethylene glycol) polymers such as Poloxamer 407. In some embodiments, a methadone formulation comprises greater than or equal to about 0.1, about 0.5, about 1, about 2, about 3, or about 4 wt % poly (ethylene glycol)-block-poly (propylene glycol)-block-poly (ethylene glycol) polymers such as Poloxamer 407.

In some embodiments, wherein a methadone formulation comprises about 50 wt % to about 60 wt % of methadone, the methadone formulation comprises about 35 wt % to about 50 wt % of polycaprolactone (PCL), such as PCL having a viscosity midpoint between about 1.5 dl/g to about 2.1 dl/g, such as Corbion PC17. In some embodiments, wherein a methadone formulation comprises about 50 wt % to about 60 wt % of methadone, the methadone formulation comprises about 0.5 wt % to about 3 wt % of poly (ethylene glycol)-block-poly (propylene glycol)-block-poly (ethylene glycol) polymers, such as H—(OCH2CH2)x—(O—CH(CH3)CH2)y—(OCH2CH2)z—OH where x and z are about 101 and y is about 56, such as Poloxamer 407.

In some embodiments, wherein a methadone formulation comprises about 56 wt % of methadone, the methadone formulation comprises about 42.5 wt. % polycaprolactone (PCL), such as PCL having a viscosity midpoint between about 1.5 dl/g to about 2.1 dl/g, such as Corbion PC17. In some embodiments, wherein a methadone formulation comprises about 56 wt % of methadone, the methadone formulation comprises about 1.5 wt % poly (ethylene glycol)-block-poly (propylene glycol)-block-poly (ethylene glycol) polymers, such as H—(OCH2CH2)x—(O—CH(CH3)CH2)y—(OCH2CH2)z—OH where x and z are about 101 and y is about 56, such as Poloxamer 407.

In some embodiments, a drug-eluting segment comprising a methadone formulation may be formed by extruding the methadone formulation. In some embodiments, the methadone formulation may be extruded to form a drug-eluting segment or component comprising a circular, triangular, wedge-or pie-shaped, or square-shaped cross-section.

Exemplary amounts of the components for some embodiments of a methadone formulation are provided in the table below. In some embodiments, a methadone formulation is used for the drug-eluting segment (and no additional formulations), these amounts correspond to the amounts of the drug-eluting segment. The amounts are given in approximate weight percent, with the understanding that when ranges are provided, the amounts are chosen so as to add up to 100%. “Pharm. accept. salt” indicates pharmaceutically acceptable salt thereof.

| | Formulation 1 | Formulation 2 | Formulation 3 |
|---|---|---|---|
| Carrier polymer-arm | | | |
| segment | | | |
| Methadone (or | 45-65 | 50-60 | 55.9 |
| pharm. accept. salt) | | | |
| PCL | 35-50 | 40-45 | 42.6 |
| P407 | 0.5-3 | 1-2 | 1.5 |

In some embodiments, a drug-eluting segment comprising a methadone formulation may release between about 5 and about 90% of the total methadone content of the drug-eluting segment within the first 24 hours of residence. In some embodiments, a drug-eluting segment comprising a methadone formulation may release greater than or equal to about 5, 10, 15, 20, 25, 30, 35, 40, 45, 50, 55, 60, 65, 70, 75, 80, or 85% of the total methadone content of the drug-eluting segment within the first 24 hours of residence. In some embodiments, a drug-eluting segment comprising a methadone formulation may release less than or equal to about 90, 85, 80, 75, 70, 65, 60, 55, 50, 45, 40, 35, 30, 25, 20, 15, or 10% of the total methadone content of the drug-eluting segment within the first 24 hours of residence. In some embodiments, a drug-eluting segment comprising a methadone formulation may release between about 5 and about 90% of the total methadone content of the drug-eluting segment after the first 24 hours of residence. In some embodiments, a drug-eluting segment comprising a methadone formulation may release greater than or equal to about 5, 10, 15, 20, 25, 30, 35, 40, 45, 50, 55, 60, 65, 70, 75, 80, or 85% of the total methadone content of the drug-eluting segment after the first 24 hours of residence. In some embodiments, a drug-eluting segment comprising a methadone formulation may release less than or equal to about 90, 85, 80, 75, 70, 65, 60, 55, 50, 45, 40, 35, 30, 25, 20, 15, or 10% of the total methadone content of the drug-eluting segment after the first 24 hours of residence. In some embodiments, a drug-eluting segment comprising a methadone formulation may release between about 5 and about 70% of the total methadone content of the drug-eluting segment after the first 48 hours of residence. In some embodiments, a drug-eluting segment comprising a methadone formulation may release greater than or equal to about 5, 10, 15, 20, 25, 30, 35, 40, 45, 50, 55, 60, or 65% of the total methadone content of the drug-eluting segment after the first 48 hours of residence. In some embodiments, a drug-eluting segment comprising a methadone formulation may release less than or equal to about 70, 65, 60, 55, 50, 45, 40, 35, 30, 25, 20, 15, or 10% of the total methadone content of the drug-eluting segment after the first 48 hours of residence. In some embodiments, a drug-eluting segment comprising a methadone formulation may release between about 5 and about 50% of the total methadone content of the drug-eluting segment after the first 72 hours of residence. In some embodiments, a drug-eluting segment comprising a methadone formulation may release greater than or equal to about 5, 10, 15, 20, 25, 30, 35, 40, or 45% of the total methadone content of the drug-eluting segment after the first 72 hours of residence. In some embodiments, a drug-eluting segment comprising a methadone formulation may release less than or equal to about 50, 45, 40, 35, 30, 25, 20, 15, or 10% of the total methadone content of the drug-eluting segment after the first 72 hours of residence. In some embodiments, a drug-eluting segment comprising a methadone formulation may release between about 5 and about 30% of the total methadone content of the drug-eluting segment after the first 96 hours of residence. In some embodiments, a drug-eluting segment comprising a methadone formulation may release greater than or equal to about 5, 10, 15, 20, or 25% of the total methadone content of the drug-eluting segment after the first 96 hours of residence. In some embodiments, a drug-eluting segment comprising a methadone formulation may release less than or equal to about 30, 25, 20, 15, or 10% of the total methadone content of the drug-eluting segment after the first 96 hours of residence. In some embodiments, a drug-eluting segment comprising a methadone formulation may release between about 5 and about 20% of the total methadone content of the drug-eluting segment after the first 120 hours of residence. In some embodiments, a drug-eluting segment comprising a methadone formulation may release greater than or equal to about 5, 10, or 15% of the total methadone content of the drug-eluting segment after the first 120 hours of residence. In some embodiments, a drug-eluting segment comprising a methadone formulation may release less than or equal to about 20, 15, or 10% of the total methadone content of the drug-eluting segment after the first 120 hours of residence.

In some embodiments, a length of a drug-eluting segment may be 1-16 mm, 10-14 mm, or 4-8 mm in length. In some embodiments, a drug-eluting segment may be less than or equal to about 16, 15, 14, 13, 12, 11, 10, 9, 8, 7, 6, 5, 4, 3, or 2 mm in length. In some embodiments, a drug-eluting segment may be greater than or equal to 1, 2, 3, 4, 5, 6, 7, 8, 9, 10, 11, 12, 13, 14, or 15 mm in length.

### Rate-Modulating Polymer Films

Release-rate modulating polymer films can be coated onto components of gastric residence systems which release agents, such as drugs. In some embodiments, a release-rate modulating polymer film controls burst release of the active ingredient(s) of a drug-eluting segment and linearizes the release of the active ingredient(s). Components coated with the release-rate modulating polymer films disclosed herein have substantially the same release-rate properties before and after exposure to heat which occurs during heat-assisted assembly of a gastric residence system. The composition, parameters, advantages, features, applications and release profiles of release-rate modulating polymer films are disclosed in International Patent Application PCT/US2020/059541, which are hereby incorporated in its entirety. In some embodiments, one or more segments of the composite arms (such as a composite arm including the drug-eluting segment or a composite arm excluding the drug- eluting segment) are coated with a release rate-modulating film. In some embodiments, the drug-eluting segment is coated with a release rate-modulating film.

In some embodiments, a drug-eluting segment of a gastric residence system may comprise 1-30 wt % release rate-modulating film. In some embodiments, a drug-eluting segment of a gastric residence system may comprise 5-15 wt % release rate-modulating film. In some embodiments, a drug-eluting segment of a gastric residence system may comprise less than or equal to 30, 25, 20, 15, 10, or 5 wt % release rate-modulating film. In some embodiments, a drug-eluting segment of a gastric residence system may comprise more than or equal to 1, 5, 10, 15, 20, or 25 wt % release rate-modulating film. In some embodiments, the amount of coating is measured as coat weight gain. The coat weight gain (cwg) is measured when applying the coating or release rate-modulating film by a scalable spray coating process. The coat weight gain is the difference between the weight of the drug-eluting segment before and after the addition of the coating (i.e., release rate-modulating film). The weight percents provided above may be measured as coat weight percent gain.

Various polymers can be used to form the release-rate modulating polymer films, including PCL. In some embodiments, the release-rate modulating polymer films comprises about 70 wt % to about 80 wt % PCL. In some embodiments, the release-rate modulating polymer films comprises about 73 wt % to about 77 wt % PCL. In some embodiments, the release-rate modulating polymer films comprises about 73.5 wt % PCL. In some embodiments, the release rate-modulating polymer film comprises less than or equal to about 80, 79, 78, 77, 76, 75, 74, 73, 72, or 71 wt % PCL. In some embodiments, the release rate-modulating polymer film comprises greater than or equal to 70, 71, 72, 73, 74, 75, 76, 77, 78, or 79 wt % PCL.

Other excipients can be added to the carrier polymers to modulate the release of agent, such as copovidone (VA64). In some embodiments, the release-rate modulating polymer films comprises about 20 wt % to about 30 wt % VA64. In some embodiments, the release-rate modulating polymer films comprises about 23 wt % to about 27 wt % VA64. In some embodiments, the release-rate modulating polymer films comprises about 24.5 wt % VA64. In some embodiments, a release rate-modulating polymer film comprises less than or equal to 30, 29, 28, 27, 26, 25, 24, 23, 22, or 21 wt % VA64. In some embodiments, a release rate-modulating polymer film comprises more than or equal to 20, 21, 22, 23, 24, 25, 26, 27, 28, or 29 wt % VA64.

In some embodiments, a release rate-modulating polymer film comprises 1-5 wt % magnesium stearate. The magnesium stearate is a process aid for the coating that prevents the arms from adhering to each other during spray coating. In some embodiments, a release rate-modulating film comprises less than or equal to 5, 4, 3, or 2 wt % magnesium stearate. In some embodiments, a release rate-modulating film comprises more than or equal to 1, 2, 3, or 4 wt % magnesium stearate.

A release rate-modulating film may be applied in a scalable spray coating process. The PCL of the coating securely adheres to the PCL-based drug polymer layers and provides a diffusion barrier to transport and prevent risk of burst release. The copovidone content may be tuned (e.g., to 24.5%) such that a coating of the proper depth (weight percent) on the drug polymer layer will support transport across the coating at a steady rate in various aqueous media. The sustained-release coating provides the rate-limiting film to transport of the drug from the drug polymer layer, thereby linearizing the rate of Methadone HCl release.

### Gastric Residence Time

The gastric residence time of the system is controlled by the degradation or weakening, or breakage, rate of the time-dependent polymeric linker in the gastric residence system. Faster degradation or weakening, or breakage of the time-dependent polymeric linker results in faster passage of the system from the stomach. The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks).

In one embodiment, the gastric residence system has a residence time between about 24 hours and about 7 days. In one embodiment, the gastric residence system has a residence time between about 48 hours and about 7 days. In one embodiment, the gastric residence system has a residence time between about 72 hours and about 7 days. In one embodiment, the gastric residence system has a residence time between about 96 hours and about 7 days. In one embodiment, the gastric residence system has a residence time between about 5 days and about 7 days. In one embodiment, the gastric residence system has a residence time between about 6 days and about 7 days.

In one embodiment, the gastric residence system has a residence time between about 24 hours and about 10 days. In one embodiment, the gastric residence system has a residence time between about 48 hours and about 10 days. In one embodiment, the gastric residence system has a residence time between about 72 hours and about 10 days. In one embodiment, the gastric residence system has a residence time between about 96 hours and about 10 days. In one embodiment, the gastric residence system has a residence time between about 5 days and about 10 days. In one embodiment, the gastric residence system has a residence time between about 6 days and about 10 days. In one embodiment, the gastric residence system has a residence time between about 7 days and about 10 days.

In one embodiment, the gastric residence system has a residence time between about 24 hours and about 14 days. In one embodiment, the gastric residence system has a residence time between about 48 hours and about 14 days. In one embodiment, the gastric residence system has a residence time between about 72 hours and about 14 days. In one embodiment, the gastric residence system has a residence time between about 96 hours and about 14 days. In one embodiment, the gastric residence system has a residence time between about 5 days and about 14 days. In one embodiment, the gastric residence system has a residence time between about 6 days and about 14 days. In one embodiment, the gastric residence system has a residence time between about 7 days and about 14 days. In one embodiment, the gastric residence system has a residence time between about 10 days and about 14 days.

The gastric residence system releases a therapeutically effective amount of agent (or salt thereof) during at least a portion of the residence time or residence period during which the system resides in the stomach. In one embodiment, the system releases a therapeutically effective amount of agent (or salt thereof) during at least about 25% of the residence time. In one embodiment, the system releases a therapeutically effective amount of agent (or salt thereof) during at least about 50% of the residence time. In one embodiment, the system releases a therapeutically effective amount of agent (or salt thereof) during at least about 60% of the residence time. In one embodiment, the system releases a therapeutically effective amount of agent (or salt thereof) during at least about 70% of the residence time. In one embodiment, the system releases a therapeutically effective amount of agent (or salt thereof) during at least about 75% of the residence time. In one embodiment, the system releases a therapeutically effective amount of agent (or salt thereof) during at least about 80% of the residence time. In one embodiment, the system releases a therapeutically effective amount of agent (or salt thereof) during at least about 85% of the residence time. In one embodiment, the system releases a therapeutically effective amount of agent (or salt thereof) during at least about 90% of the residence time. In one embodiment, the system releases a therapeutically effective amount of agent (or salt thereof) during at least about 95% of the residence time. In one embodiment, the system releases a therapeutically effective amount of agent (or salt thereof) during at least about 98% of the residence time. In one embodiment, the system releases a therapeutically effective amount of agent (or salt thereof) during at least about 99% of the residence time.

### Gastric Residence Systems Comprising Methadone

In some embodiments, a stellate-shaped dosage form for administration of methadone can comprise arms, which arms in turn comprise 1) a carrier polymer-agent arm segment; 2) one or more enteric linkers; 3) one or more time-dependent linkers; 4) an inactive segment; 5) one or more radioactive (inert) segments; and/or 5) other optional spacers. The arms are connected to an elastomeric core in a stellate device arrangement. Typically, six arms are used for a stellate dosage form. In some embodiments, wherein six arms are used for a stellate dosage form, any one of 1, 2, 3, 4, 5, or 6 arms comprise a carrier polymer-agent arm segment. In some embodiments, wherein six arms are used for a stellate dosage form, 3 arms comprise the carrier polymer-agent arm segment. In some embodiments, wherein six arms are used for a stellate dosage form, 6 arms comprise the carrier polymer-agent arm segment. In some embodiments, wherein six arms are used for a stellate dosage form, 2 arms comprise the carrier polymer-agent arm segment. In some embodiments, wherein six arms are used for a stellate dosage form, 4 arms comprise the carrier polymer-agent arm segment.

The carrier polymer-agent arm segments of the methadone dosage form can comprise methadone (or a pharmaceutically acceptable salt thereof), polycaprolactone, and poloxamer 407 (P407). In some embodiments, typically six arms are used for a stellate dosage form, and either 1, 2, 3, 4, 5 or 6 of the arms comprise the carrier polymer-agent arm segment. In some embodiments, 3 of the arms comprise the carrier polymer-agent arm segment. In some embodiments, 6 of the arms comprise the carrier polymer-agent arm segment. In some embodiments, 2 of the arms can comprises the carrier polymer-agent arm segment. In some embodiments, 4 of the arms can comprises the carrier polymer-agent arm segment. In some embodiments, arms that do not comprises a carrier agent-polymer segment comprise an inactive segment instead. In some embodiments, the total amount of agent contained in the dosage form is 1, 2, 3, 4, 5, or 6 times the amount of agent contained in a single arm. In some embodiments, the total amount of agent contained in the dosage form is 3 times the amount of agent contained in a single arm. In some embodiments, the total amount of agent contained in the dosage form is 6 times the amount of agent contained in a single arm. In some embodiments, the total amount of agent contained in the dosage form is 4 times the amount of agent contained in a single arm. The total amount of weight of methadone, pharmaceutically acceptable salt of methadone, or salt of methadone in the stellate dosage form can range from 10 mg to about 320 mg of methadone. In some embodiments, a dosage form for administration of methadone comprises a gastric residence system comprising about 50 mg to about 300 mg of methadone. In some embodiments, a dosage form for administration of methadone comprises a gastric residence system comprising less than or equal to about 320, 300, 280, 260, 240, 220, 200, 180, 160, 140, 120, 100, 80, 60, 40, or 20 mg of methadone. In some embodiments, a dosage form for administration of methadone comprises a gastric residence system comprising greater than or equal to about 10, 20, 40, 60, 80, 100, 120, 140, 160, 180, 200, 220, 240, 260, 280, or 300 mg methadone.

The inert arm segments of the dosage form can comprise polycaprolactone (PCL), a radiopaque substance, and optionally coloring. The polycaprolactone used can be from about 1.5 dL/g to about 1.9 dL/g viscosity, such as about 1.7 dL/g. The radiopaque substance can be (BiO)2CO3. Any pharmaceutically acceptable coloring agent can be used. An example of coloring that can be used includes FD&C Blue #5.

The inactive arm segments (e.g., non-radioactive) may comprise polycaprolactone, copovidone (VA64), a poloxamer (P407), and an optional coloring.

The enteric disintegrating matrices of the dosage form can comprise polycaprolactone (PCL), hydroxypropyl methyl cellulose acetate succinate (HPMCAS), poloxamer 407 (P407), and optionally coloring. The polycaprolactone used can be from about 1.5 dL/g to about 1.9 dL/g viscosity, such as about 1.7 dL/g. The HPMCAS used can be MG grade (M grade: about 7-11% acetyl content, about 10-14% succinoyl content, about 21-25% methoxyl content, about 5-9% hydroxypropoxy content; G grade: granular). Any pharmaceutically acceptable coloring agent can be used. An example of coloring that can be used includes ferrosoferric oxide.

The time dependent disintegrating matrices of the methadone dosage form can comprise poly (D,L-lactide-co-glycolide) (PLGA), polyethylene oxide (PEO), and optionally coloring. The poly (D,L-lactide-co-glycolide) can be in about a 75:25 lactide: glycolide molar ratio with a viscosity range of about 0.32-0.44 dL/g. The polyethylene oxide used can be from about 60,000 MW to about 125,000 MW, such as about 90,000 MW to 110,000 MW, or about 100,000 MW.

The central elastomer of the methadone dosage form can be of about 40 A to about 60 A durometer, such as about 45 A to about 55 A durometer, or about 50 A durometer. The central elastomer can be made from liquid silicone rubber; e.g., the central elastomer can comprise cured liquid silicone rubber.

In some embodiments, an assembled arm of a methadone gastric residence dosage form may be attached to the central elastomer at a polymeric linker segment. The polymeric linker segment can serve as an attachment point between the assembled arm and the central elastomer. In some embodiments, the polymeric linker segment may attach the central elastomer to a time-dependent disintegrating matrix segment of the arm. In some embodiments, the polymeric linker comprises polycaprolactone such as PCL having a viscosity midpoint between about 1.5 dl/g to about 2.1 dl/g, such as Corbion PC17. In some embodiments, the polymeric linker is adjacent to the central elastomer.

In some embodiments, the gastric residence system further comprises a release rate-modulating film comprising about 73.5 wt % of polycaprolactone (PCL), such as PCL having a viscosity midpoint between about 1.5 dl/g to about 2.1 dl/g, such as Corbion PC17. In some embodiments, the release rate-modulating film further comprises about 24.5 wt % of copovidone, such as VA64. In some embodiments, the release rate-modulating film further comprises about 2.0 wt % of Mg stearate.

Exemplary amounts for the various components of the dosage form are provided in the table below. The amounts are given in approximate weight percent, with the understanding that when ranges are provided, the amounts are chosen so as to add up to 100%.

| | Formulation 1 | Formulation 2 | Formulation 3 |
|---|---|---|---|
| Carrier polymer- | | | |
| arm segment | | | |
| Methadone (or | 45-65 | 50-60 | 55.9 |
| pharm. accept. salt) | | | |
| PCL | 35-50 | 40-45 | 42.6 |
| P407 | 0.5-3 | 1-2 | 1.5 |
| Inactive spacer | | | |
| PCL | 61-71 | 64-69 | 66.45 |
| VA64 | 27-37 | 30-34 | 32 |
| P407 | 0.2-4 | 0.5-2.5 | 1.5 |
| coloring (optional) | 0.005-0.2 | 0.01-0.1 | 0.05 (e.g. |
| | | | Blue #1) |
| Enteric | | | |
| disintegrating | | | |
| matrix | | | |
| PCL | 30-40 | 32-37 | 33.95 |
| HPMCAS | 60-70 | 62-66 | 63.95 |
| P407 | 0.5-5 | 1-3 | 2 |
| coloring (optional) | 0.01-0.5 | 0.05-0.15 | 0.1 (e.g. |
| | | | E172) |
| Time-dependent | | | |
| disintegrating | | | |
| matrix | | | |
| PCL | 40-50 | 43-47 | 44.95 |
| PDLG5004A | 30-40 | 33-37 | 35 |
| PDLG5004 | 10-25 | 15-20 | 18 |
| PEO(100k) | 0.5-5 | 1-3 | 2 |
| coloring (optional) | 0.005-0.2 | 0.01-0.1 | 0.05 (e.g. |
| | | | E172) |

The assembled arms can comprise 1) a polymeric linker segment; 2) a first disintegrating matrix segment; 3) a second disintegrating segment; 4) a first inactive segment; and 5) a drug eluting segment, wherein the drug eluting segment comprises a carrier polymer and methadone or a salt thereof, which can be arranged in various orders. One such order is, starting from the proximal end which is attached to the central elastomer, and proceeding to the distal end: (a polymeric linker segment) (a first disintegrating matrix segment) (a second disintegrating segment) (a first inactive segment) (a drug eluting segment).

In some embodiments, one or more arms of a gastric residence system may not comprise a drug-eluting segment. For example, an assembled arm without a drug-eluting segment can comprise 1) a polymeric linker segment; 2) a first disintegrating matrix segment; 3) a second disintegrating segment; and 4) a first inactive segment, which can be arranged in various orders. One such order is, starting from the proximal end which is attached to the central elastomer, and proceeding to the distal end: (a polymeric linker segment) (a first disintegrating matrix segment) (a first inactive segment) (a second disintegrating segment) (a first inactive segment).

Approximate dimensions for the length of the segments on each arm are provided below. Optional rPCL spacers (inert segments) of about 0.2-2 mm width, such as about 0.5 mm width, can be inserted between any two components below, or added to the outer tip of the assembled arm, or between the inner tip of the assembled arm and the elastomeric core.

| | Dimension | | Dimension | | Dimension | |
|---|---|---|---|---|---|---|
| Component | set 1 | | set 2 | | set 3 | |
| Carrier polymer- | 9-14 | mm | 11-12 | mm | 11.8 | mm |
| agent segment | | | | | | |
| Inactive segment | 1-4 | mm | 2-3 | mm | 2.5 | mm |
| (used with drug- | | | | | | |
| eluting arm) | | | | | | |
| Inactive segment | 12-16 | mm | 13-15 | mm | 14 | mm |
| (used with non-drug- | | | | | | |
| eluting arm) | | | | | | |
| Timed disintegrating | 0.25-5 | mm | 0.5-2 | mm | 1 | mm |
| matrix (First | | | | | | |
| disintegrating | | | | | | |
| segment) | | | | | | |
| Enteric | 0.25-5 | mm | 0.5-2 | mm | 1 | mm |
| disintegrating matrix | | | | | | |
| (Second | | | | | | |
| disintegrating | | | | | | |
| segment) | | | | | | |
| Polymeric Linker | 0.5-2.5 | mm | 1-1.5 | mm | 1-1.5 | mm |
| Segment | | | | | | |

### Exemplary Gastric Residence Systems

The following gastric residence systems are exemplary to better illustrate certain embodiments of the system described herein. As these examples are only exemplary, they are not intended to limit the gastric residence system described herein. One skilled in the art, in view of the provided disclosure, would be able to contemplate additional configurations of the gastric residence system.

In some embodiments, the gastric residence system comprises at least one arm including a drug-eluting segment, wherein the arm comprises: (a) a polymeric linker segment as described in any of the embodiments above; (b) a timed disintegrating matrix as described in any of the embodiments above, (c) a first inert segment as described in any of the embodiments of inert segment above, (d) an enteric disintegrating matrix as described in any of the embodiments above, (e) a first inactive segment as described in any of the embodiments of inactive segment above, and (f) a drug-eluting segment as described in any of the embodiments described above. The polymeric linker segment can be attached to a central elastomer. In some embodiments, a second inert segment may be located between the enteric disintegrating segment and the first inactive segment.

In some embodiments, the gastric residence system comprises at least one arm including a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a first inactive segment, and (f) a drug-eluting segment, wherein:
the central elastomer comprises liquid silicone rubber (LSR) having a hardness of about 40 to about 65 durometer;
   (a) the polymeric linker segment comprises 100 wt % PCL;
   (b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
   (c) the first inert segment comprises about 65 wt % to about 75 wt % PCL, and about 25 wt % to about 35 wt % (BiO)2CO3;
   (d) the enteric disintegrating matrix comprises about 60 wt % to about 70 wt % HPMCAS, about 30 wt % to about 40 wt % PCL, and about 0.5 wt % to about 5 wt % poloxamer (such as P407) and optionally about 0.01 wt % to about 0.5 wt % iron oxide (such as E172);
   (e) the first inactive segment comprises about 60 wt % to about 70 wt % PCL, and about 25 wt % to about 40 wt % of VA64, about 0.5 wt % to about 2.5 wt % of P407, and about 0.005 wt % to about 0.2 wt % of a pigment or coloring;
   (f) the drug-eluting segment comprises about 50 wt % to about 60 wt % of methadone; about 35 wt % to about 50 wt % of PCL, about 0.5 wt % to about 5 wt % of P407.

In some embodiments, the gastric residence system comprises at least one arm including a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a first inactive segment, and (f) a drug-eluting segment, wherein:
the central elastomer comprises liquid silicone rubber (LSR) having a hardness of about 45 to about 55 durometer;
   (a) the polymeric linker segment comprises 100 wt % PCL;
   (b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
   (c) the first inert segment comprises about 68 wt % to about 72 wt % PCL, and about 28 wt % to about 32 wt % (BiO)2CO3;
   (d) the enteric disintegrating matrix comprises about 62 wt % to about 66 wt % HPMCAS, about 32 wt % to about 37 wt % PCL, and about 1 wt % to about 3 wt % poloxamer (such as P407) and optionally about 0.05 wt % to about 0.15 wt % iron oxide (such as E172);
   (e) the first inactive segment comprises about 62 wt % to about 68 wt % PCL, and about 30 wt % to about 35 wt % of VA64, about 1 wt % to about 2 wt % of P407, and about 0.01 wt % to about 0.1 wt % of a pigment or coloring;
   (f) the drug-eluting segment comprises about 52 wt % to about 58 wt % of methadone; about 40 wt % to about 45 wt % of PCL, about 1 wt % to about 2 wt % of P407.

In some embodiments, the gastric residence system comprises at least one arm including a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a first inactive segment, and (f) a drug-eluting segment, wherein:
the central elastomer comprises liquid silicone rubber (LSR) having a hardness of about 50 durometer;
   (a) the polymeric linker segment comprises 100 wt % PCL;
   (b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
   (c) the first inert segment comprises about 68 wt % to about 72 wt % PCL, and about 28 wt % to about 32 wt % (BiO)2CO3;
   (d) the enteric disintegrating matrix comprises about 63.95 wt % HPMCAS, about 33.95 wt % PCL, and about 2 wt % poloxamer (such as P407) and optionally about 0.1 wt % iron oxide (such as E172);
   (e) the first inactive segment comprises about 66.45 wt % PCL, and about 32 wt % of VA64, about 1.5 wt % of P407, and about 0.05 wt % of a pigment or coloring;
   (f) the drug-eluting segment comprises about 55.9 wt % of methadone; about 42.6 wt % of PCL, about 1.5 wt % of P407.

In some embodiments, the gastric residence system comprises at least one arm excluding a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a first inactive segment, wherein:
the central elastomer comprises liquid silicone rubber (LSR) having a hardness of about 40 to about 65 durometer;
   (a) the polymeric linker segment comprises 100 wt % PCL;
   (b) the timed disintegrating matrix comprises about 40 wt % to about 50 wt % PCL, about 30 wt % to about 40 wt % of acid terminated copolymer of DL-lactide and glycolide (50/50 molar ratio) having a viscosity midpoint of about 0.4 dl/g, about 10 wt % to about 25 wt % of copolymer of DL-lactide and glycolide (50/50 molar ratio) having a viscosity midpoint of about 0.4 dl/g, about 0.5 wt % to about 5 wt % of polyethylene glycol 100 k, and about 0.005 wt % to about 0.2 wt % color-absorbing dye E172;
   (c) the first inert segment comprises about 65 wt % to about 75 wt % PCL, and about 25 wt % to about 35 wt % (BiO)2CO3;
   (d) the enteric disintegrating matrix comprises about 60 wt % to about 70 wt % HPMCAS, about 30 wt % to about 40 wt % PCL, and about 0.5 wt % to about 5 wt % poloxamer (such as P407) and optionally about 0.01 wt % to about 0.5 wt % iron oxide (such as E172);
   (e) the first inactive segment comprises about 61 wt % to about 71 wt % of PCL, about 27 wt % to about 37 wt % of VA64, about 0.2 wt % to about 4 wt % of P407, and about 0.005 wt % to about 0.2 wt % of a pigment or coloring.

In some embodiments, the gastric residence system comprises at least one arm excluding a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a first inactive segment, wherein:
the central elastomer comprises liquid silicone rubber (LSR) having a hardness of about 45 to about 55 durometer;
   (a) the polymeric linker segment comprises 100 wt % PCL;
   (b) the time-dependent disintegrating matrix comprises about 43 wt % to about 47 wt % PCL, about 33 wt % to about 37 wt % of acid terminated copolymer of DL-lactide and glycolide (50/50 molar ratio) having a viscosity midpoint of about 0.4 dl/g, about 15 wt % to about 20 wt % of copolymer of DL-lactide and glycolide (50/50 molar ratio) having a viscosity midpoint of about 0.4 dl/g, about 1 wt % to about 3 wt % of polyethylene glycol 100 k, and about 0.01 wt % to about 0.1 wt % color-absorbing dye E172;
   (c) the first inert segment comprises about 68 wt % to about 72 wt % PCL, and about 28 wt % to about 32 wt % (BiO)2CO3;
   (d) the enteric disintegrating matrix comprises about 62 wt % to about 66 wt % HPMCAS, about 32 wt % to about 37 wt % PCL, and about 1 wt % to about 3 wt % poloxamer (such as P407) and optionally about 0.05 wt % to about 0.15 wt % iron oxide (such as E172);
   (e) the first inactive segment comprises about 64 wt % to about 69 wt % of PCL, about 30 wt % to about 34 wt % of VA64, about 0.5 wt % to about 2.5 wt % of P407, and about 0.01 wt % to about 0.1 wt % of a pigment or coloring.

In some embodiments, the gastric residence system comprises at least one arm excluding a drug eluting segment, wherein the arm can be attached to a central elastomer, and the arm comprises one or more of: (a) a polymeric linker segment; (b) a timed disintegrating matrix, (c) a first inert segment, (d) an enteric disintegrating matrix, (e) a first inactive segment, wherein:
the central elastomer comprises liquid silicone rubber (LSR) having a hardness of about 50 durometer;
   (a) the polymeric linker segment comprises 100 wt % PCL;
   (b) time-dependent disintegrating matrix, the time-dependent disintegrating matrix comprises about 44.95 wt % PCL, about 35 wt % of acid terminated copolymer of DL-lactide and glycolide (50/50 molar ratio) having a viscosity midpoint of about 0.4 dl/g, about 18 wt % of copolymer of DL-lactide and glycolide (50/50 molar ratio) having a viscosity midpoint of about 0.4 dl/g, about 2 wt % of polyethylene glycol 100 k and about 0.05 wt % color-absorbing dye E172;
   (c) the first inert segment comprises about 70 wt % PCL, and about 30 wt % (BiO)2CO3;
   (d) the enteric disintegrating matrix comprises about 63.95 wt % HPMCAS, about 33.95 wt % PCL, and about 2 wt % poloxamer (such as P407) and about 0.1 wt % iron oxide (such as E172);
   (e) the first inactive segment comprises about 66.45 wt % of PCL, about 32 wt % of VA64, about 1.5 wt % of P407, and about 0.05 wt % of a pigment or coloring.

In any of the above-described embodiments, the arm can be attached to the central elastomer at the polymeric linker segment. That is, the polymeric linker segment is the proximal end of the arm.

In some embodiments, the dosage form for administration of methadone comprises a gastric residence system, wherein the gastric residence system comprises between one and five inactive segments (e.g., one per arm, for a total of one to five arms comprising an inactive segment). In some embodiments, the gastric residence system comprises a first inactive segment comprising about 66.45 wt % of polycaprolactone (PCL), such as PCL having a viscosity midpoint between about 1.5 dl/g to about 2.1 dl/g, such as Corbion PC17. In some embodiments, the gastric residence system comprises a first inactive segment comprising about, about 32.0 wt % of copovidone, such as VA64. In some embodiments, the gastric residence system comprises a first inactive segment comprising about 1.5 wt % of poly (ethylene glycol)-block-poly (propylene glycol)-block-poly (ethylene glycol) polymers, such as H—(OCH2CH2)x—(O—CH (CH3)CH2)y—(OCH2CH2)z—OH where x and z are about 101 and y is about 56, such as Poloxamer 407 (P407). In some embodiments, the gastric residence system comprises a first inactive segment comprising about 0.005 wt % of iron oxide, such as E172.

In some embodiments, a gastric residence system dosage form for administration of one or more agents can comprise a radiopaque segment, where the segment comprises about 70 wt % of polycaprolactone (PCL), such as PCL having a viscosity midpoint between about 1.5 dl/g to about 2.1 dl/g, such as Corbion PC17. In some embodiments, the gastric residence system comprises a radiopaque segment comprising about 30 wt % of (BiO)2CO. In some embodiments, the gastric residence system comprises a radiopaque (inert) segment comprising about 70 wt % of Corbion PC17, and about 30 wt % of (BiO)2CO3.

In some embodiments, a gastric residence system dosage form for administration of methadone comprises a central elastomer. In some embodiments, the gastric residence system dosage form comprises a drug-eluting segment comprising about 46.6 mg methadone per drug-eluting arm. In some embodiments, the gastric residence system dosage form comprises a drug-eluting segment comprising about 16.09 mg methadone per drug-eluting arm. In some embodiments, the gastric residence system further comprises a release rate-modulating film comprising about 73.5 wt % of polycaprolactone (PCL), such as PCL having a viscosity midpoint between about 1.5 dl/g to about 2.1 dl/g, such as Corbion PC17. In some embodiments, the release rate-modulating film further comprises about 24.5 wt % of copovidone, such as VA64. In some embodiments, the release rate-modulating film also comprises about 2 wt % magnesium stearate. In some embodiments, the gastric residence system further comprises a time-dependent disintegrating matrix comprising about 44.95 wt % of polycaprolactone (PCL), such as PCL having a viscosity midpoint between about 1.5 dl/g to about 2.1 dl/g, such as Corbion PC17. In some embodiments, the time-dependent disintegrating matrix further comprises about 35.0 wt % of an acid terminated copolymer of DL-lactide and glycolide (50/50 molar ratio) having a viscosity midpoint between about 0.32 dl/g to about 0.48 dl/g (such as about 0.4 dl/g), such as PDLG 5004A. In some embodiments, the time-dependent disintegrating matrix further comprises about 18.0 wt % of a copolymer of DL-lactide and glycolide (50/50 molar ratio) having a viscosity midpoint between about 0.32 dl/g to about 0.48 dl/g (such as about 0.4 dl/g), such as PDLG 5004. In some embodiments, the time-dependent disintegrating matrix further comprises about 2.0 wt % of polyethylene glycol, such as polyethylene glycol with average molecular weight of 100,000, such as PEO100K. In some embodiments, the time-dependent disintegrating matrix further comprises about 0.05 wt % of iron oxide, such as E172. In some embodiments, the gastric residence system further comprises a pH-dependent disintegrating matrix comprising about 33.95 wt % of polycaprolactone (PCL), such as PCL having a viscosity midpoint between about 1.5 dl/g to about 2.1 dl/g, such as Corbion PC17. In some embodiments, the pH-dependent disintegrating matrix further comprises about 63.95 wt % of hypromellose acetate succinate, such as HPMCAS-MG. In some embodiments, the pH-dependent disintegrating matrix further comprises about 2.0 wt % of poly (ethylene glycol)-block-poly (propylene glycol)-block-poly (ethylene glycol) polymers, such as H—(OCH2CH2)x—(O—CH(CH3)CH2)y—(OCH2CH2)z—OH where x and z are about 101 and y is about 56, such as Poloxamer 407 (P407). In some embodiments, the pH-dependent disintegrating matrix further comprises about 0.1 wt % of iron oxide, such as E172. In some embodiments, the gastric residence system further comprises one or more inactive segments. In some embodiments, the gastric residence system further comprises a radiopaque segment comprising about 70 wt % of polycaprolactone (PCL), such as PCL having a viscosity midpoint between about 1.5 dl/g to about 2.1 dl/g, such as Corbion PC17. In some embodiments, the radiopaque segment comprises about 30 wt % of (BiO)2CO3. In some embodiments, the gastric residence system further comprises one or more polymeric linker segments comprising about 100 wt % of polycaprolactone (PCL). In some embodiments, a polymeric linker segment is located at the proximal end of the arm, immediately adjacent to the central elastomer.

In some embodiments, the gastric residence system has one arm comprising a drug-eluting segment and five arms not comprising a drug-eluting segment. In some embodiments, the gastric residence system has two arms comprising a drug-eluting segment and four arms not comprising a drug-eluting segment. In some embodiments, the gastric residence system has three arms comprising a drug-eluting segment and three arms not comprising a drug eluting segment. In some embodiments, the gastric residence system has four arms comprising a drug-eluting segment and two arms not comprising a drug-eluting segment. In some embodiment, the gastric residence system has five arms comprising a drug-eluting segment and one arm not comprising a drug-eluting segment. In some embodiments, the gastric residence system has six arms comprising a drug-eluting segment.

### Central Elastomer

The central elastomer provides the gastric residence system with the ability to be compacted into a compressed configuration, which can be placed in a capsule or other suitable containing structure for administration to a subject.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 45 to about 60 durometer.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 45 to about 55 durometer.

In some embodiments, a dosage form for administration of one or more agents comprises a gastric residence system, wherein the gastric residence system comprises a central elastomer comprising a liquid silicone rubber (LSR). In some embodiments, the LSR has a hardness of about 50 durometer.

### EXAMPLES

The disclosure is further illustrated by the following non-limiting examples.

### Example 1: Methadone Dosage Form (Extended-Release Gastric Residence System)

In this Example, a dosage form according to the present invention includes a gastric residence system, the gastric residence system is formulated to include methadone.

The gastric residence system includes a central elastomer that provides the gastric residence system with the ability to be compacted into a compressed configuration. The gastric residence system illustrated in this Example is another different arrangement of the “star” configuration. In an example of the methadone-formulated gastric residence system, the stellate contains 6 arms each comprising a drug-eluting segment.

FIG. 3 is labelled to show the various elements of this configuration. The system 300 comprises a central elastomeric core 310 which is in the shape of an “asterisk” having six short branches. A segment 380 of the arm is attached to one short asterisk branch. The segment 380 is followed by a segment 360, a segment 370, a segment 350, a segment 330, and a segment 340 in sequence.

The gastric residence system has an average size of about 46 mm and each segment has a length ranging from about 0.5 mm to about 11.8 mm. The table below provides a listing of the length of each segment of an active arm (i.e., an arm comprising a drug-eluting segment) in the gastric residence system. Each range or value below can be considered to be “about” the range or value indicated, or exactly the range or value indicated.

| Segment | Length | |
|---|---|---|
| 330 | 2.5 | mm |
| 340 | 11.8 | mm |
| 350 | 1.0 | mm |
| 360 | 1.0 | mm |
| 370 | 0.5 | mm |
| 380 | 1-1.5 | mm |

The central elastomeric core 310 comprises a liquid silicone rubber (LSR) having a hardness of 50 durometer.

In this example, the dosage form provided here contains 6 arms each comprising a drug-eluting segment, wherein the dosage form comprises about 280 mg of methadone for administration. Methadone is included in a carrier polymer-agent segment 340 (e.g., a drug-eluting segment). The drug-eluting segment comprises about 55.9 wt % of methadone, about 42.6 wt % of Corbion PC17, and about 1.5 wt % of P407. Also contemplated in the present application are variations of this dosage form with increased numbers and/or lengths of the drug-eluting segments to achieve higher doses of the drug, for example, methadone.

The gastric residence system further includes a time-dependent disintegrating matrix or linker, referred as the segment 360, as well as a pH-dependent disintegrating matrix or linker, referred as the segment 350. In addition, the gastric residence system includes a structural segment 370, and a polymeric linker segment 380.

The time-dependent disintegrating matrix (segment 360) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 350) comprises about 63.95 wt % HPMCAS, about 33.95 wt % Corbion PC17, about 2 wt % P407 and about 0.1 wt % color-absorbing dye E172. The structural segment 370 can be a radiopaque-PCL segment, comprising about 70 wt % PCL, and about 30 wt % (BiO)2CO3.

The inactive segment (segment 430) comprises about 66.45 wt % of Corbion PC17, about 32.0 wt % of VA 64, about 1.5 wt % of P407 and about 0.05 wt % of FD&C Blue 1 Aluminum lake.

Segment 380, at the proximal end of each arm, is a polymeric linker segment comprising about 100 wt % PCL.

In some embodiments, each drug arm is coated by a release rate-modulating film. Specifically, the coating comprises about 73.5 wt % of Corbion PC17, about 24.5 wt % of VA64 and about 2% of Magnesium Stearate, and is applied in an amount of about 5-20% of the pre-coating weight of the segment. In some embodiments, the release rate-modulating film may be applied in an amount of less than or equal to 20, 18, 16, 14, 12, 10, 8, or 6% of the pre-coating weight of the segment. In some embodiments, the release rate-modulating film may be applied in an amount that is greater than or equal to 5, 6, 8, 10, 12, 14, 16, or 18% of the pre-coating weight of the segment.

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the methadone-formulated gastric residence system, the stellate contains 4 arms each comprising a drug-eluting segment, and 2 arms not comprising a drug-eluting segment. Also contemplated in this application are other gastric residence systems containing 6 arms of which either 1, 2, 3, 4, 5, to 6 arms comprise a drug-eluting segment.

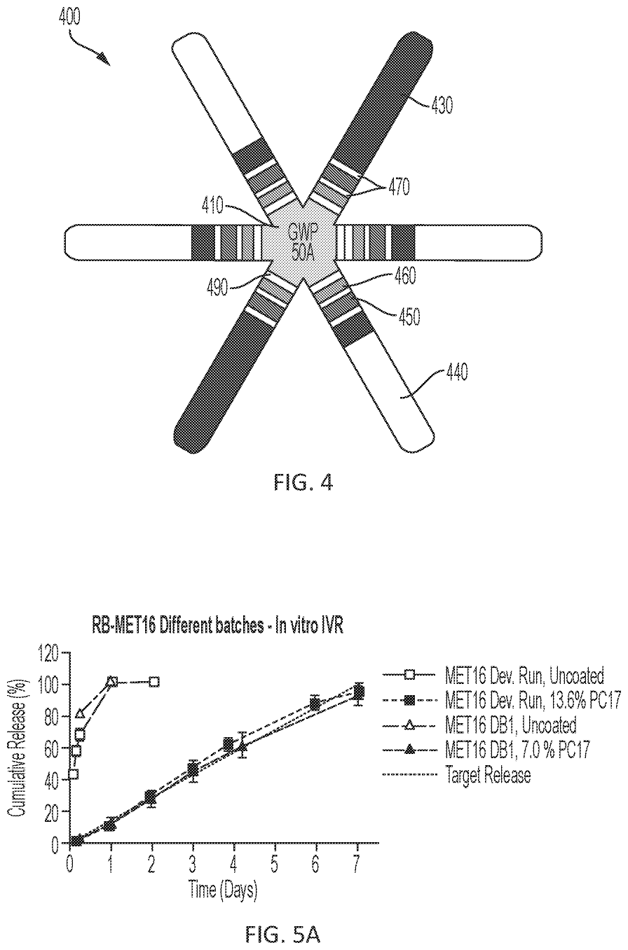

FIG. 4 is labelled to show the various elements of this configuration. The system 400 comprises a central elastomeric core 410 which is in the shape of an “asterisk” having six short branches.

For an arm containing a drug-eluting segment, a segment 480 of the arm is attached to one short asterisk branch. The segment 480 is followed by a segment 460, a first segment 470, a segment 450, a second segment 470, a segment 430, and a segment 440 in sequence.

For an arm not containing a drug-eluting segment, a segment 480 of the arm is attached to one short asterisk branch. The segment 480 is followed by a segment 460, a first segment 470, a segment 450, a second segment 470, and a segment 430 in sequence.

The gastric residence system has an average size of about 46 mm and each segment has a length ranging from about 0.5 mm to about 14 mm. The table below provides a listing of the length of each segment in the gastric residence system. Each range or value below can be considered to be “about” the range or value indicated, or exactly the range or value indicated.

| Segment | Length | |
|---|---|---|
| 430 (drug- | 2.5 | mm |
| eluting) | | |
| 430 (non- | 14 | mm |
| drug- | | |
| eluting) | | |
| 440 | 11.8 | mm |
| 450 | 1.0 | mm |
| 460 | 1.0 | mm |
| 470 | 0.5 | mm |
| 480 | 1-1.5 | mm |

The central elastomeric core 410 comprises a liquid silicone rubber (LSR) having a hardness of 50 durometer.

In this example, the dosage form provided here contains 6 arms, four of which comprise a drug-eluting segment, wherein the dosage form comprises about 187 mg of methadone for administration. In some embodiments, each drug-eluting segment may comprise about 46.7 mg methadone HCl. In some embodiments, each drug-eluting segment may comprise about 46.5 mg levomethadone HCl. Methadone is included in a carrier polymer-agent segment 440 (e.g., a drug-eluting segment). The drug-eluting segment comprises about 55.9 wt % of methadone, about 42.6 wt % of Corbion PC17, and about 1.5 wt % of P407. Also contemplated in the present application are variations of this dosage form with increased numbers and/or lengths of the drug-eluting segments to achieve higher doses of the drug, for example, methadone.

The two arms that are drug-free comprise a larger inactive segment (segment 430) than that of the drug-eluting segments. The inactive segment 430 each comprises about 66.45 wt % of Corbion PC17, about 32.0 wt % of VA 64, about 1.5 wt % of P407 and about 0.05 wt % of FD&C Blue 1 Aluminum lake.

The gastric residence system further includes a time-dependent disintegrating matrix or linker, referred as the segment 460, as well as a pH-dependent disintegrating matrix or linker, referred as the segment 450. In addition, the gastric residence system includes a structural segment 470.

The time-dependent disintegrating matrix (segment 460) comprises about 44.95 wt % Corbion PC17, about 35 wt % of acid terminated copolymer of DL-lactide and glycolide (PDLG5004A), about 18 wt % of copolymer of DL-lactide and glycolide (PDLG5004), about 2 wt % of polyethylene glycol 100 k and about 0.05 wt % color-absorbing dye E172. The pH-dependent disintegrating matrix (segment 450) comprises about 63.95 wt % HPMCAS, about 33.95 wt % Corbion PC17, about 2 wt % P407 and about 0.1 wt % color-absorbing dye E172 (ferrosoferric oxide). The structural segment 470 can be a radiopaque-PCL segment, comprising about 70 wt % PCL, and about 30 wt % (BiO)2CO3.

Segment 480, at the proximal end of each arm, is a polymeric linker segment comprising about 100 wt % PCL.

In some embodiments, each drug arm is coated by a release rate-modulating film. Specifically, the coating comprises about 73.5 wt % of Corbion PC17, about 24.5 wt % of VA64 and about 2% of Magnesium Stearate, and is applied in an amount of about 5-20% of the pre-coating weight of the segment. In some embodiments, the release rate-modulating film may be applied in an amount of less than or equal to 20, 18, 16, 14, 12, 10, 8, or 6% of the pre-coating weight of the segment. In some embodiments, the release rate-modulating film may be applied in an amount that is greater than or equal to 5, 6, 8, 10, 12, 14, 16, or 18% of the pre-coating weight of the segment.

The gastric residence system is assembled and then placed into an appropriate sized capsule as described in Example 1 of International Patent Application PCT/US2020/059541. The dosage form described here differs from a gastric residence system previously described in International Patent Application No. PCT/US2020/059541, and other gastric residence systems previously designated as LYN-005.

In another example of the methadone-formulated gastric residence system, the stellate contains 2 arms each comprising a drug-eluting segment, and 4 arms not comprising a drug-eluting segment. Also contemplated in this application are other gastric residence systems containing 6 arms of which either 1, 2, 3, 4, 5, to 6 arms comprise a drug-eluting segment.

### Example 2: Methadone Dosage Form (Extended-Release Gastric Residence System) with Four Arms Comprising Drug-Eluting Segments

In this second Example of a methadone dosage form, the dosage form includes a gastric residence system, and the gastric residence system is formulated to include methadone. Unlike the dosage form of Example 1, however, the dosage form of the instant example includes six arms total, with only four of them each comprising a drug-eluting segment. In this example, the dosage form comprises a 187 mg dose of methadone.

The gastric residence system of Example 2 includes a central elastomer that provides the gastric residence system with the ability to be compacted into a compressed configuration.

The gastric residence system of FIG. 4 more closely resembles that comprising a 187 mg dose of methadone (as opposed to that of FIG. 3). FIG. 4 is labelled to show the various elements of this configuration. The system 400 comprises a central elastomeric core 410 which is in the shape of an “asterisk” having six short branches.

For an arm containing a drug-eluting segment, a segment 480 of the arm is attached to one short asterisk branch. The segment 480 is followed by a segment 460, a first segment 470, a segment 450, a second segment 470, a segment 430, and a segment 440 in sequence.

For an arm not containing a drug-eluting segment, a segment 480 of the arm is attached to one short asterisk branch. The segment 480 is followed by a segment 460, a first segment 470, a segment 450, a second segment 470, and a segment 430 in sequence. Each of segments 480, 460, 470, 450, and 430 correspond to segments 380, 360, 370, 350, and 330 of FIG. 3.

The gastric residence system is assembled and a sleeve (size 0 capsule, transparent, body only) is placed over the gathered distal ends of the stellate arms (when the stellate is in its compressed configuration). Once sleeved, the sleeved gastric residence system is encapsulated with a size 00EL capsule, white opaque (2% TiO₂).

In another example of the methadone-formulated gastric residence system, the stellate contains 2 arms each comprising a drug-eluting segment, and 4 arms not comprising a drug-eluting segment. Also contemplated in this application are other gastric residence systems containing 6 arms of which either 1, 2, 3, 4, 5, to 6 arms comprise a drug-eluting segment.

### Example 3: Evaluating Methadone Release for Coated and Uncoated Dosage Forms

FIGS. 5A and 5B show methadone release profiles for different batches of gastric residence systems comprising the same methadone formulation. Specifically, the methadone combination formulation used in the dosage forms is as follows:

| Component | Wt. % |
|---|---|
| Methadone | 55.9 |
| PCL | 42.8 |
| P407 | 1.5 |

Additionally, the gastric residence systems in this trial all comprised arms having a wedge-shaped cross-sectional area. The drug-eluting components of each trial run were as follows:

| | | | Linear density | |
|---|---|---|---|---|
| Formulation | Extruder | API % | (mg/mm) | Coating |
| MET16 Dev. | 16 mm, | 55.9 | 7.20 | PC17 |
| Run | double pass | | | |
| MET16 DB1 | 16 mm, | 55.9 | 5.73 | PC17 |
| | double pass | | | |

The coating, PC17, comprises 73.5 wt % PCL, 24.5 wt % VA64, and 2 wt % magnesium stearate.

As shown in FIG. 5A, the coated dosage forms can control burst release and slow the methadone release from the drug-eluting segments. Further, FIG. 5B shows that for MET16 DB1, a 7.0% PC17 coating linearized the release (relative to the target release), whereas for MET16 Dev Run, the cumulative release linearization occurs with 13.6% PC17 coating.

### Example 4: Comparing Release Rate-Modulating Film Coat Weights

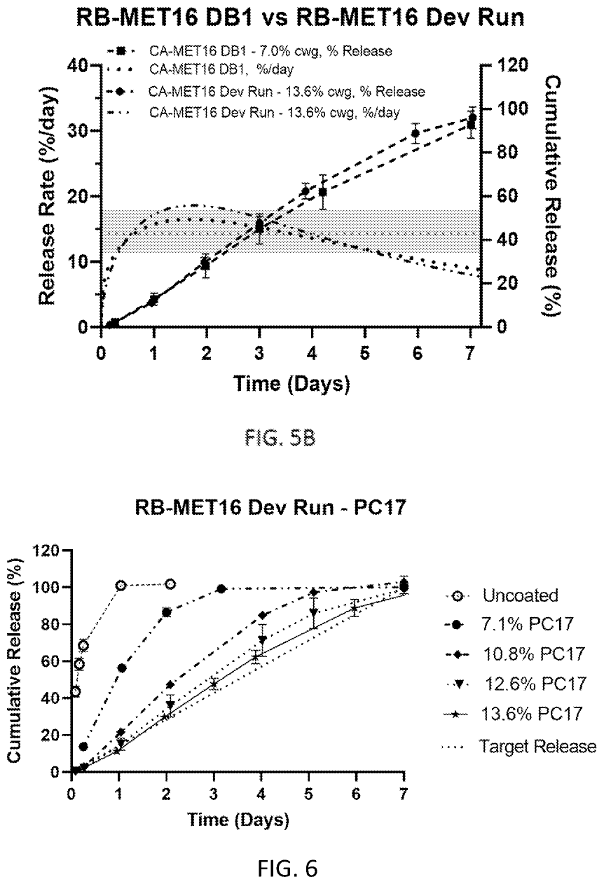

FIG. 6 shows release profiles for gastric residence dosage forms comprising methadone and various coat weights of a release rate-modulating film. As shown, FIG. 6 shows the progression of the linearization of the cumulative release of Methadone HCl when coated 7.1%, 10.8%, 12.6%, and 13.3% weight percent of PC17 coating. The coating, PC17, comprises 73.5 wt % PCL, 24.5 wt % VA64, and 2 wt % magnesium stearate. The best linearized cumulative release profile (relative to the target release) is obtained for a MET16 matrix coated with 13.6% PC17. The MET16 formulation of the tested gastric residence systems is provided in the table below.

| Component | Wt. % |
|---|---|
| Methadone | 55.9 |
| PCL | 42.8 |
| P407 | 1.5 |

### Example 5: Fed-Fasted Media Study

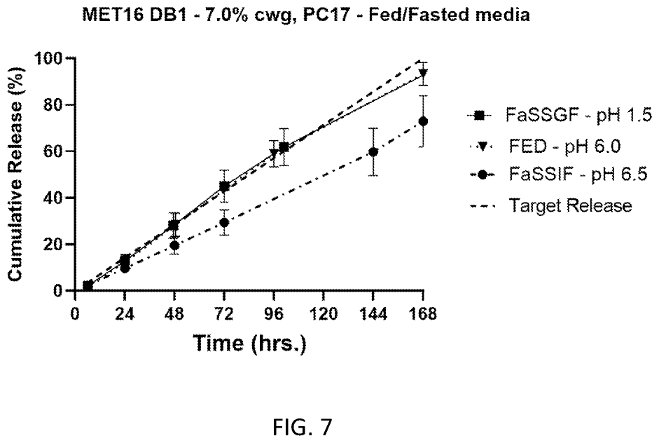

FIG. 7 shows methadone release profiles gastric residence systems in different simulated gastric fluids. As shown, fasted state simulated gastric fluid (FaSSGF), FED, and fasted state intestinal fluid (FaSSIF) were tested. The gastric residence systems tested included the methadone formulation described above in Examples 2 and 3. The drug-eluting segments were coated with 7 coat weight percent gain (cwg) release rate-modulating film. The coating, PC17, comprised 73.5 wt % PCL, 24.5 wt % VA64, and 2 wt % magnesium stearate.

### Example 6: 0 & 3-Day EtOH Challenge

The methadone release was tested for gastric residence systems comprising methadone in the presence of ethanol. The gastric residence systems tested include the methadone and release rate-modulating film described in Examples 2, 3, and 4 above.

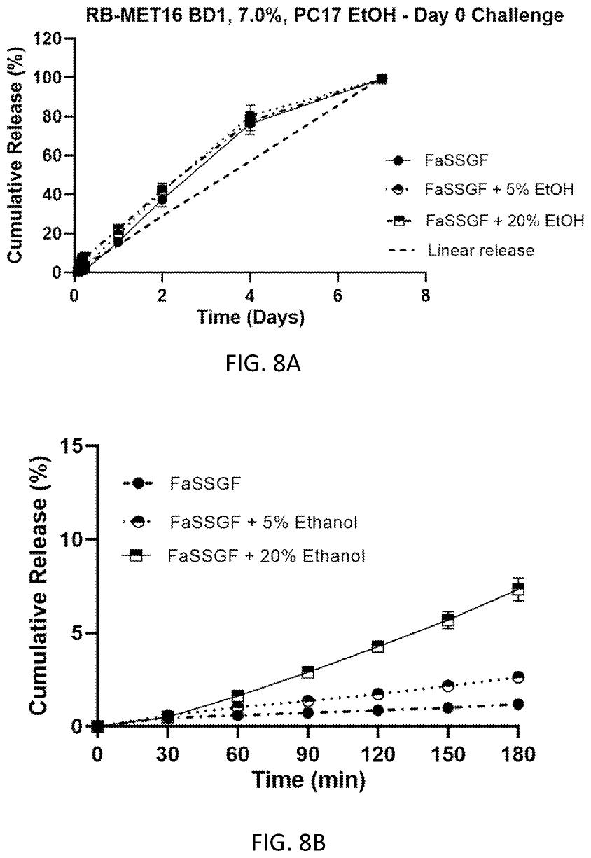

FIGS. 8A and 8B show the results of the day 0 ethanol challenge. During this challenge, samples were subjected to an initial FaSSGF incubation for 2 hours. The samples were then subjected to FaSSGF with 5 or 20% EtOH. The media were sampled every 30 minutes for 2 hours and replenished with their respective EtOH-spiked FaSSGF media. After the challenge, the media was switched back to FaSSGF. Aliquots were subsequently taken at 6-h, 24-h, 48-h, 96-h and 168-h timepoints.

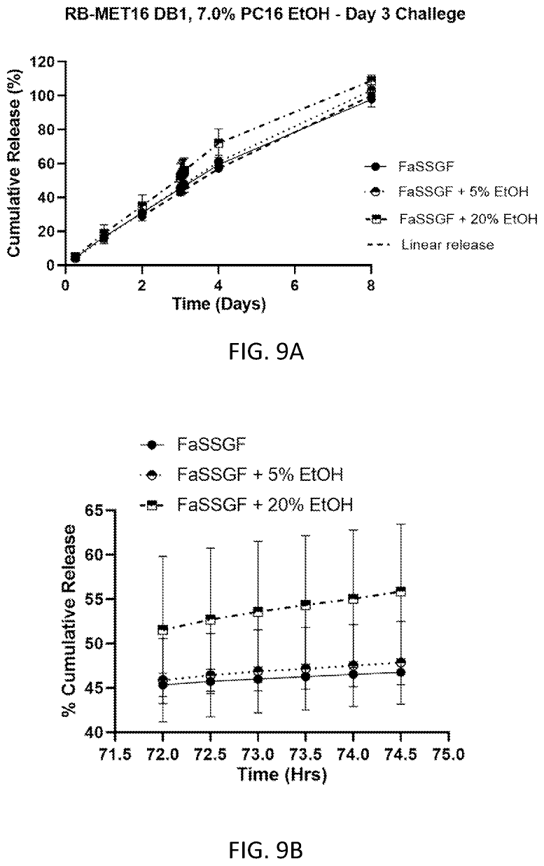

FIGS. 9A and 9B show the results of the day 3 ethanol challenge. From the first day of this challenge, samples were subjected to an initial FaSSGF incubation for 3 days. Aliquots were taken at 6-h, 24-h and 48-h timepoints. On the third day, the aliquot was pulled at 72-h timepoint, and the media was changed to FaSSGF spiked with either 5 or 20% EtOH for a total duration of 2 hours. The media was sampled every 30 minutes and replenished with their respective EtOH-spiked FaSSGF media. After the challenge, the media was switched back to FaSSGF. Aliquots were subsequently taken at 96-h and 168-h timepoints.

### Example 7: Beagle Study

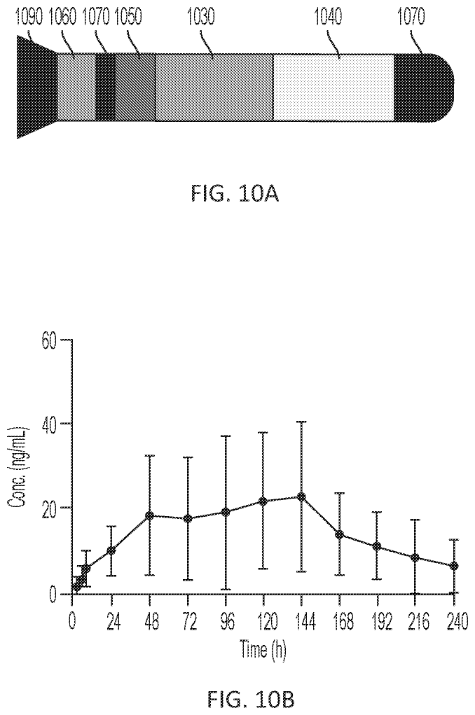

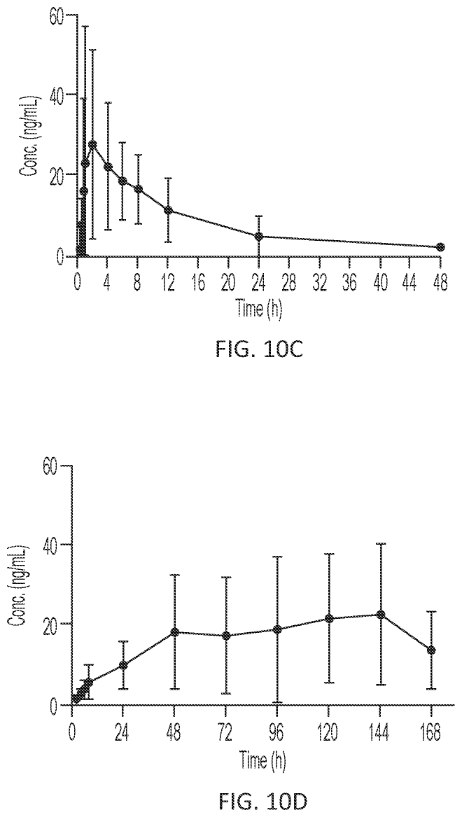

The release of methadone administrated with a gastric residence dosage form was studied in beagles, as described below. The results are provided in FIGS. 10B-10D. Male beagle dogs were fasted overnight prior to dose administration.

A 10 mg tablet of Methadone HCl was orally administered on Study Day 1 and Study Day 8. For the administration on Study Day 8, dogs were treated with a 50 mg fluconazole tablet 12 hours before and 12 hours after the dose. (Methadone is rapidly metabolized in dog, fluconazole (CYP3A4 inhibitor) was administered to reduce this metabolism and enable adequate plasma methadone quantification. IR data presented in the report is from Study Day 8.) Plasma samples were collected at pre-dose, 0.25, 0.5, 0.75, 1, 2, 4, 6, 8, 12, 24 and 48 hours post-dose.

LYN-014-M containing 100 mg methadone HCl, was orally administered on Study Day 22 to dogs that had been treated with fluconazole as described above and then daily through Study Day 30. Plasma samples were collected at pre-dose, 2, 4, 6, 8, 24, 48, 72, 96, 120, 144, 168, 192, 216 and 240 hours post-dose. An arm of a LYN-014-M gastric residence system is shown in FIG. 10A (wherein 1090 represents polycaprolactone segment, 1060 represents a time-dependent disintegrating matrix segment comprising, 1070 represents a radiopaque-PCL segment, 1050 represents a pH-dependent disintegrating matrix segment, 1030 represents and inactive segment, and 1040 represents a carrier polymer-agent segment). As shown in the Figure, the drug-eluting segment comprises 16.09 mg methadone, and the arm comprises an inactive segment at a distal end (connected to the drug-eluting MET 16-RB) segment. Further, the arm of the stellate comprises a polymeric linking member (PCL) on a proximal end connected directly to the central elastomer (not shown), a time-disintegrating matrix segment connected to the polymeric linker, an inert segment connected to the time-disintegrating matrix segment, an enteric disintegrating segment connected to the inert segment, an inactive segment connected to the inert segment, a drug-eluting segment connected to the inactive segment, and an inert segment connected to the drug-eluting segment.

Sample extracts were prepared by protein precipitation from plasma and methadone concentrations were measured using LC/MS/MS. Pharmacokinetic parameters were calculated by noncompartmental analysis.

Mean plasma PK parameters for Methadone HCl (10 mg) and LYN-014-M (100 mg of methadone) and individual plasma PK parameters are presented in the tables below. As shown, following a single oral administration of a 10 mg methadone IR tablet, methadone was rapidly absorbed with Tmax achieved 1 to 8 hours post dose. Mean systemic exposure was 32.0 ng/ml and 317 h*ng/mL for Cmax and AUCtau, respectively, and the mean half-life was 8 h.

Following a single oral administration of 100 mg LYN-014-M, the Cmax (29.5 ng/mL) was delayed (Tmax 24 to 144 h) compared to the IR (Tmax 1 to 8 h). The dose normalized AUCtau of LYN-014-M dosage was comparable to that of the IR (mean 28.2 h*ng/ml and 31.7 h*ng/mL for the LYN-014 and IR forms, respectively).

Thus, single oral (PO) administration of a LYN-014-M confirmed measurable plasma concentrations of methadone for 7+ days after dosing in dog. When compared to a single dose of methadone HCl IR tablets, LYN-014-M had a similar exposure as assessed by Cmax and AUC.

Methadone PK Parameters Following Single Dose Administration of IR Tablets and LYN-014-M Capsules in Beagle Dogs (LYN-262-MET)

| | Methadone | | | | | | | | | |
|---|---|---|---|---|---|---|---|---|---|---|
| Dosage | HCL Dose | | Tmax\hat{ } | Tlast\hat{ } | Cmax | Half-life | AUClast | AUCinf | AUCtau | AUCtau/D |
| Form | (mg) | ROA | (hr) | (hr) | (ng/mL) | (hr) | (hr · ng/mL) | (hr · ng/mL) | (hr · ng/mL) | (hr · ng/mL) |
| IR | 10 | po | 2 | 24 | 32.0 ± | 8.19 ± | 351 ± | 389 ± | 317 ± | 31.7 ± |
| tablet | | | | | 29.7 | 2.03 | 311 | 312 | 228 | 22.8 |
| LYN-014-M | 100 | po | 108 | 240 | 29.5 ± | NR | 3510 ± | 4400 ± | 2820 ± | 28.2 ± |
| capsule | | | | | 19.8 | | 2450 | 3430 | 1940 | 19.4 |

Data are presented as mean ± standard deviation
\hat{ }Tmax = median,
NR = not reported

| Dosage | Dose | | Half-life | Tmax\hat{ } | Tlast\hat{ } | Cmax | AUClast | AUCinf | AUCtau | AUCtau/D | Cavg |
|---|---|---|---|---|---|---|---|---|---|---|---|
| Form | (mg) | Animal | (h) | (h) | (h) | (ng/mL) | (h*ng/mL) | (h*ng/mL) | (h*ng/mL) | (h*ng/mL/mg) | (ng/mL) |
| IR | 10 | 1006 | 7.21 | 8 | 24 | 11.3 | 161 | 186 | 161 | 16.1 | 6.69 |
| | | 1007 | 10.1 | 4 | 24 | 16.3 | 248 | 320 | 248 | 24.8 | 10.3 |
| | | 1008 | 9.01 | 2 | 24 | 20.1 | 248 | 301 | 248 | 24.8 | 10.3 |
| | | 1009 | 6.65 | 2 | 24 | 31.2 | 280 | 307 | 280 | 28 | 11.7 |
| | | 1010 | 5.54 | 2 | 24 | 21.9 | 192 | 203 | 192 | 19.2 | 7.99 |
| | | 1011 | 10.6 | 1 | 48 | 91.1 | 980 | 1020 | 775 | 77.5 | 32.3 |
| | | Mean | 8.19 | 2 | 24 | 32.0 | 351 | 389 | 317 | 31.7 | 13.2 |
| | | SD | 2.03 | — | — | 29.7 | 311 | 312 | 228 | 22.8 | 9.51 |
| | | CV % | 24.8 | — | — | 92.9 | 88.5 | 80.4 | 72 | 72 | 72 |
| | | Geomean | 7.97 | — | — | 24.8 | 283 | 323 | 273 | 27.3 | 11.4 |
| LYN- | 100 | 1006 | 25.5 | 120 | 216 | 34.4 | 3130 | 3250 | 2640 | 26.4 | 15.7 |
| 014-M | | 1007 | 83.7 | 144 | 240 | 13.6 | 2250 | 3080 | 1640 | 16.4 | 9.73 |
| | | 1008 | 17.3 | 144 | 240 | 51.9 | 4810 | 4850 | 4210 | 42.1 | 25 |
| | | 1009 | 125 | 48 | 240 | 17.1 | 2240 | 3330 | 1800 | 18 | 10.7 |
| | | 1010 | 38.6 | 24 | 240 | 7.45 | 894 | 972 | 692 | 6.92 | 4.12 |
| | | 1011 | 134 | 96 | 240 | 52.7 | 7760 | 10900 | 5970 | 59.7 | 35.5 |
| | | Mean | 70.8 | 108 | 240 | 29.5 | 3510 | 4400 | 2820 | 28.2 | 16.8 |
| | | SD | 51.3 | — | — | 19.8 | 2450 | 3430 | 1940 | 19.4 | 11.6 |
| | | CV % | 72.4 | — | — | 67 | 69.6 | 78 | 68.7 | 68.7 | 68.7 |
| | | Geomean | 53.7 | — | — | 23.4 | 2840 | 3460 | 2270 | 22.7 | 13.5 |

\hat{ }= median

The table below shows the presence of the gastric residence system in each of six dogs (1006-1011) through the duration of the study. “Day” (i.e., Day 1-28) are the days counting with Day 1 being the day the stellate was administered. “Study day” (i.e., Study day 22-49) is counting starting from the day the first IR methadone dose was administered. The numbers in the “Broken” cells refer to the number of arms still attached to the stellate in the stomach.

| | Study Day | | | | | | | | | | | | | | | |
|---|---|---|---|---|---|---|---|---|---|---|---|---|---|---|---|---|
| | 22 | 23 | 24 | 25 | 26 | 27 | 28 | 29 | 31 | 35 | 37 | 39 | 42 | 44 | 46 | 49 |
| | Day | | | | | | | | | | | | | | | |
| # | 1 | 2 | 3 | 4 | 5 | 6 | 7 | 8 | 10 | 14 | 16 | 18 | 21 | 23 | 25 | 28 |
| 1006 | I | I | I | I | B | B | B | B | NS | NS | NS | NS | NS | NS | NS | NS |
| | | | | | (5) | (5) | (5) | (5) | | | | | | | | |
| 1007 | I | I | I | I | I | B | B | B | B | NA | B | B | B | NS | NS | NS |
| | | | | | | (5) | (5) | (5) | (5) | | (4) | (4) | (4) | | | |
| 1008 | I | I | B | B | NA | B | B | NA | B | NA | B | NS | NS | NS | NS | NS |
| | | | (5) | (5) | | (5) | (4) | | (4) | | (5) | | | | | |
| 1009 | I | I | I | I | I | I | I | I | I | B | B | B | B | B | B | NS |
| | | | | | | | | | | (5) | (5) | (4) | (4) | (4) | (3) | |
| 1010 | I | I | I | I | I | I | I | I | I | B | B | B | NS | NS | NS | NS |
| | | | | | | | | | | (4) | (3) | (3) | | | | |
| 1011 | I | I | I | I | I | B | B | B | B | NA | B | B | B | B | NS | NS |
| | | | | | | (5) | (5) | (4) | (2) | | (2) | (2) | (2) | (2) | | |

Key
I = Intact
B = Broken
P = Passing
NS = Not Seen
NA = Blurry but resident

FIG. 10B shows plasma concentrations versus time for single oral dose of the LYN-014-M (100 mg of methadone) over 10 days.

Plasma concentrations versus time for a single oral dose of Methadone HCl (10 mg) is shown in FIG. 10C.

Plasma concentrations versus time for single oral dose of the LYN-014-M (100 mg of methadone) over 7 days is shown in FIG. 10D.

### Example 7: Measuring Release Profile using Assay and Purity Method

Gastric residence systems comprising levomethadone hydrochloride 187 mg ER capsules were tested for assay and purity following manufacture and repeated after 3 and 6 months storage at 25° C./60% RH as well as after 3 months storage at 30° C./65% RH. The testing method used were consistent with the “Assay and Purity” method described below in the Release Profile Testing Methods section. Levomethadone hydrochloride content at initial testing was 100% of label claim (187 mg). No significant change was found at 3 or 6 months. There were no detectable levomethadone hydrochloride degradants at initial or at any stability time point, reported at not more than 0.1%/not detected. Release profile of levomethadone hydrochloride from the drug product remained controlled with no burst at all timepoints.

| | Assay | Degradation Productions |
|---|---|---|
| Acceptance | 85-115% of Label | Individual Unspecified |
| Criteria | Claim | Degradation |
| | | Products: NMT 0.5% each |
| | | Total Degradation Products: |
| | | NMT 2.0% |
| T = 0/Initial | Average = 100% | |
| 3 Months @ | Average = 101% | No degradants detected |
| 25 C./60% RH | | above reporting limit, |
| 3 Months @ | Average = 102% | Not more than 0.1% |
| 30 C./65% RH | | PASS |
| 6 Months @ | Average = 101% | |
| 25 C./60% RH | | |

### Example 8: Measuring Release Profile Using Chiral Purity Method

Gastric residence system comprising levomethadone hydrochloride 187 mg ER capsules were tested for chiral purity following manufacture and repeated after 3 and 6 months storage at 25° C./60% RH as well as after 3 months storage at 30° C./65% RH. The testing method used were consistent with the “Chiral Purity” method described below in the Release Profile Testing Methods section. The amount of the opposite enantiomer, dextromethadone, present remained constant at 0.2%. This level is observed in the levomethadone hydrochloride drug substance purchased and does not change during manufacture or on stability.

| | Chiral Purity |
|---|---|
| Acceptance | Dextromethadone |
| Criteria | enantiomer: NMT |
| | 1.0% |
| T = 0/Initial | Average = 0.2% |
| 3 Months @ | |
| 25 C./60% RH | |
| 3 Months @ | |
| 30 C./65% RH | |
| 6 Months @ | |
| 25 C./60% RH | |

### Example 9: Measuring Release Profile Using Dissolution Method

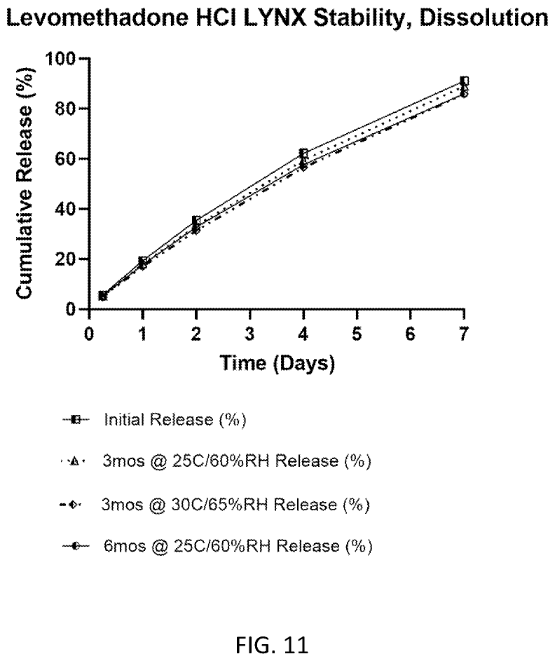

Dissolution was performed on gastric residence systems comprising levomethadone hydrochloride 187 mg ER capsules following manufacture and repeated after 3 and 6 months storage at 25° C./60% RH as well as after 3 months storage at 30° C./65% RH. The testing method used were consistent with the “Dissolution” method described below in the Release Profile Testing Methods section. As shown in FIG. 11, no statistically significant trend was found. Release profile of levomethadone hydrochloride from the drug product remained controlled with no burst at all timepoints.

### Example 10: Measuring Release Profile Using In Vitro Release Method

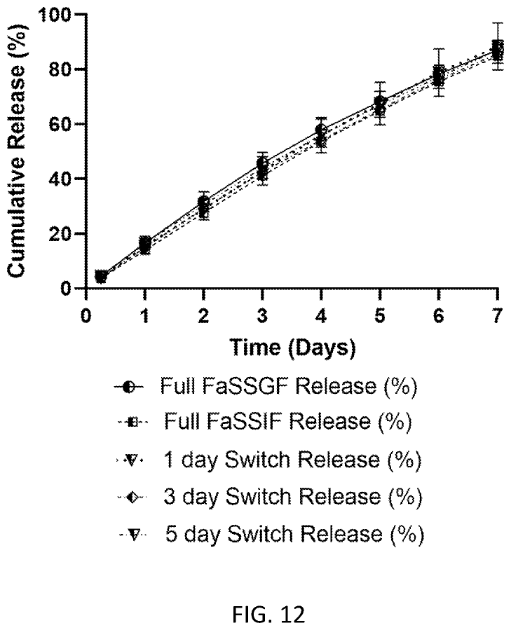

In Vitro Release testing according to the “In Vitro Release (IVR)” testing method described below in the Release Profile Testing Methods section was performed on coated intermediates to assess the release profile in the gastric environment and determine impact of the dosage form passing into the intestine early while continuing to release drug. The study was performed by starting in a biorelevant media to simulate fasted gastric state (FaSSGF) and then transferring the sample to a media that represents fasted intestinal state (FaSSIF) after 1, 3, or 5 days. As shown in FIG. 12, no significant change in release profile was observed.

### Release Profile Testing Methods

Assay and Purity: The portion of the gastric residence system or drug product containing the API (e.g., levomethadone hydrochloride) is cut/removed and dissolved in stabilized tetrahydrofuran. Ethanol is added as a counter-solvent to obtain a 5% tetrahydrofuran 95% ethanol solution containing the API (e.g., levomethadone). The solution is diluted as necessary and filtered. Samples are analyzed for levomethadone hydrochloride content and degradation products by HPLC according to the method conditions in the table below. The method has a limit of detection of 0.1% of nominal levomethadone peak area.

| Column | 2.1 mm × 100 mm; 2.5 μm Phenyl-Hexyl Column | | | | | | |
|---|---|---|---|---|---|---|---|
| Column | 40° C. | | | | | | |
| Temperature | | | | | | | |
| Detection | 230 nm | | | | | | |
| Flow Rate | 0.35 mL/min | | | | | | |
| Injection Volume | 3.0 μL | | | | | | |
| Mobile Phase A | 0.1% Trifluoroacetic Acid in Water | | | | | | |
| Mobile Phase B | Acetonitrile with 0.1% Trifluoroacetic Acid | | | | | | |
| | Time | | | | | | |
| Gradient | (min) | 0.0 | 5.0 | 25.0 | 30.0 | 31.0 | 35.0 |
| | % A | 95 | 95 | 0 | 0 | 95 | 95 |
| | % B | 5 | 5 | 100 | 100 | 5 | 5 |

Chiral Purity (method adapted from Ph. Eur. monograph): The portion of the gastric residence system (or drug product) containing the API (e.g., levomethadone hydrochloride) is cut/removed from the drug product and dissolved in stabilized tetrahydrofuran. Mobile phase is added as a counter-solvent to obtain a 5% tetrahydrofuran 95% mobile phase solution containing API (e.g., levomethadone). The solution is diluted as necessary and filtered. Samples are analyzed by chiral HPLC to confirm API (e.g., levomethadone) identity and quantify presence of opposite enantiomer relative to API (e.g., levomethadone) content. The HPLC method conditions are presented in the following table:

| | |
|---|---|
| Column | Astec Cyclobond \| 2000 SP Column 5 μm, 4.6 mm × |
| | 250 mm |
| Column | 10° C. |
| Temperature | |
| Detection | 210 nm |
| Flow Rate | 0.7 mL/min |
| Injection Volume | 10 μL |
| Mobile Phase | 85/15 0.1M Potassium dihydrogen phosphate with 1% |
| (isocratic) | trimethylamine, pH 4.0/Acetonitrile, v/v |
| Run Time | 25 mins |

Dissolution: Dissolution performed on levomethadone drug products were completed in accordance with USP <711> using the dissolution parameters described in the table below.

Dissolution Parameters

| | |
|---|---|
| Dissolution Apparatus | USP <711> Dissolution, Apparatus 2 |
| | (with sinker) |
| Media | 0.025N hydrochloric acid |
| Media Volume | 900 mL |
| Temperature | 37.0 ± 0.5° C. |
| Paddle Rotation Speed | 50 rpm |
| Sampling Volume | 5.0 mL |

Samples are pulled over the course of 1 week and analyzed by HPLC using the conditions below.

HPLC Parameters

| | |
|---|---|
| Column | 2.1 mm × 100 mm; 2.5 μm Phenyl-Hexyl |
| | Column |
| Column Temperature | 30° C. |
| Detection | 230 nm |
| Flow Rate | 0.4 mL/min |
| Injection Volume | 5.0 μL |
| Mobile Phase A | 0.1% Trifluoroacetic Acid in Water |
| Mobile Phase B | Acetonitrile with 0.1% Trifluoroacetic Acid |
| Mobile Phase Composition | 70% Mobile Phase A/30% Mobile Phase B |
| (isocratic) | |
| Run Time | 5 Mins |

In Vitro Release (IVR): IVR is performed on levomethadone intermediates in various biorelevant media. The term “intermediate” refers to a drug-loaded polymer segment (coated or uncoated), and not an entire gastric residence system. One drug-containing polymeric sample is added to 30 mL of biorelevant media and stationed in a shaking incubator set to 37° C. and 200 rpm. Samples are pulled over the course of 2-7 days and sampled for HPLC analysis (method conditions below). The sample is transferred to 30 mL of fresh media and returned to shaking incubator for continued testing.

In Vitro Release Parameters

| | |
|---|---|
| Setup | Incubated shaker containing 40-mL |
| | vials |
| Media | Varius biorelevant media (e.g. |
| | FaSSGF: Fasted State Simulated |
| | Gastric Fluid) |
| Media Volume | 30 mL |
| Temperature | 37.0° C. |
| Shaker Speed | 200 rpm |
| Sampling Volume | Full media replacement |

HPLC Parameters

| | |
|---|---|
| Column | 2.1 mm × 100 mm; 2.5 μm Phenyl-Hexyl |
| | Column |
| Column Temperature | 30° C. |
| Detection | 230 nm |
| Flow Rate | 0.4 mL/min |
| Injection Volume | 5.0 μL |
| Mobile Phase A | 0.1% Trifluoroacetic Acid in Water |
| Mobile Phase B | Acetonitrile with 0.1% Trifluoroacetic Acid |
| Mobile Phase Composition | 70% Mobile Phase A/30% Mobile Phase B |
| (isocratic) | |
| Run Time | 5 Mins |

### EMBODIMENTS

Embodiment 1. A gastric residence system comprising: at least one drug-eluting component comprising methadone or a salt thereof, 35-50 wt % polycaprolactone, and 0.5-3 wt % poloxamer; and a release rate-modulating film coating the at least one drug-eluting component, wherein the gastric residence system is configured to be maintained within a stomach of a human body for at least 48 hours and to release methadone for at least 48 hours, and the at least one drug-eluting component with the release rate-modulating film is configured to release at least 10% of the methadone or the salt thereof after the first 24 hours of residence within the stomach.

Embodiment 2. The gastric residence system of embodiment 1, comprising 50-60 wt % racemic methadone.

Embodiment 3. The gastric residence system of embodiment 1, comprising 50-60 wt % levomethadone.

Embodiment 4. The gastric residence system of any of embodiments 1-3, comprising 1-2 wt % poloxamer.

Embodiment 5. The gastric residence system of any of embodiments 1-4, wherein the poloxamer comprises P407.

Embodiment 6. The gastric residence system of any of embodiments 1-5, wherein the release rate-modulating film comprises polycaprolactone, copovidone, and magnesium stearate.

Embodiment 7. The gastric residence system of embodiment 6, wherein the release rate-modulating film comprises 60-90 wt % polycaprolactone.

Embodiment 8. The gastric residence system of embodiment 6 or 7, wherein the release rate-modulating film comprises 70-75 wt % polycaprolactone.

Embodiment 9. The gastric residence system of any of embodiments 6-8, wherein the release rate-modulating film comprises 10-40 wt % copovidone.

Embodiment 10. The gastric residence system of any of embodiments 6-9, wherein the release rate-modulating film comprises 20-30 wt % copovidone.

Embodiment 11. The gastric residence system of any of embodiments 6-10, wherein the release rate-modulating film comprises 1-5 wt % magnesium stearate.

Embodiment 12. The gastric residence system of any of embodiments 6-11, wherein the release rate-modulating film comprises 1-3 wt % magnesium stearate.

Embodiment 13. The gastric residence system of any of embodiments 1-12, wherein the at least one drug-eluting component comprises 20 mg to 50 mg of racemic methadone or a salt thereof.

Embodiment 14. The gastric residence system of any of embodiments 1-12, wherein the at least one drug-eluting component comprises 20 mg to 50 mg of levomethadone or a salt thereof.

Embodiment 15. The gastric residence system of any of embodiments 1-14, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

Embodiment 16. The gastric residence system of embodiment 15, wherein the plurality of arms comprises six arms.

Embodiment 17. The gastric residence system of embodiment 15 or 16, wherein at least two arms of the plurality of arms comprises a drug-eluting component of the at least one drug-eluting component.

Embodiment 18. The gastric residence system of embodiment 15 or 16, wherein at least three arms of the plurality of arms comprises a drug-eluting component of the at least one drug-eluting component.

Embodiment 19. The gastric residence system of embodiment 15 or 16, wherein six arms of the plurality of arms comprises a drug-eluting component of the at least one drug-eluting component.

Embodiment 20. The gastric residence system of any of embodiments 15-19, wherein each arm of the plurality of arms comprises a polymeric linker segment attached to the central elastomer, the polymeric linker segment comprising polycaprolactone.

Embodiment 21. The gastric residence system of embodiment 20, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

Embodiment 22. The gastric residence system of embodiment 21, wherein each arm of the plurality of arms comprises a first inert segment attached to the first disintegrating matrix segment, the first inert segment comprising polycaprolactone and (BiO)₂CO₃.

Embodiment 23. The gastric residence system of embodiment 22, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

Embodiment 24. The gastric residence system of embodiment 23, wherein each arm of the plurality of arms comprises an inactive segment attached to the second disintegrating matrix segment, the inactive segment comprising polycaprolactone, copovidone, and a poloxamer.

Embodiment 25. The gastric residence system of embodiment 24, wherein a drug-eluting arm of the plurality of arms comprises the drug-eluting component attached to the inactive segment.

Embodiment 26. The gastric residence system of any of embodiments 1-25, wherein the area under the curve of the gastric residence system is between 1000 and 6000 hr·ng/mL.

Embodiment 27. A method of treating an opioid abuse disorder in an individual, comprising administering the gastric residence system of any one of embodiments 1-26 to the individual.

Embodiment 28. A method of making a gastric residence system comprising: extruding at least one drug-eluting component comprising methadone or a salt thereof, 35-50 wt % polycaprolactone, and 0.5-3 wt % poloxamer; and applying a release rate-modulating film to the at least one drug-eluting component, wherein the gastric residence system is configured to be maintained within a stomach of a human body for at least 48 hours and to release methadone for at least 48 hours, and the at least one drug-eluting component with the release rate-modulating film is configured to release at least 10% of the methadone or the salt thereof after the first 24 hours of residence within the stomach.

Embodiment 29. The method of embodiment 28, wherein the gastric residence system comprises 50-60 wt % racemic methadone.

Embodiment 30. The method of embodiment 28, wherein the gastric residence system comprises 50-60 wt % levomethadone.

Embodiment 31. The method of any of embodiments 28-30, wherein the gastric residence system comprises 1-2 wt % poloxamer.

Embodiment 32. The method of any of embodiments 28-31, wherein the poloxamer comprises P407.

Embodiment 33. The method of any of embodiments 28-32, wherein the release rate-modulating film comprises polycaprolactone, copovidone, and magnesium stearate.

Embodiment 34. The method of embodiment 33, wherein the release rate-modulating film comprises 60-90 wt % polycaprolactone.

Embodiment 35. The method of embodiment 33 or 34, wherein the release rate-modulating film comprises 70-75 wt % polycaprolactone.

Embodiment 36. The method of any of embodiments 33-35, wherein the release rate-modulating film comprises 10-40 wt % copovidone.

Embodiment 37. The method of any of embodiments 33-36, wherein the release rate-modulating film comprises 20-30 wt % copovidone.

Embodiment 38. The method of any of embodiments 33-37, wherein the release rate-modulating film comprises 1-5 wt % magnesium stearate.

Embodiment 39. The method of any of embodiments 33-38, wherein the release rate-modulating film comprises 1-3 wt % magnesium stearate.

Embodiment 40. The method of any of claims 28-39, wherein the at least one drug-eluting component comprises 20 mg to 50 mg of racemic methadone or a salt thereof.

Embodiment 41. The method of any of embodiments 28-39, wherein the at least one drug-eluting component comprises 20 mg to 50 mg of levomethadone or a salt thereof.

Embodiment 42. The method of any of embodiments 28-41, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

Embodiment 43. The method of embodiment 42, wherein the plurality of arms comprises six arms.

Embodiment 44. The method of embodiment 42 or 43, wherein at least two arms of the plurality of arms comprises a drug-eluting component of the at least one drug-eluting component.

Embodiment 45. The method of embodiment 42 or 43, wherein at least three arms of the plurality of arms comprises a drug-eluting component of the at least one drug-eluting component.

Embodiment 46. The method of embodiment 42 or 43, wherein six arms of the plurality of arms comprises a drug-eluting component of the at least one drug-eluting component.

Embodiment 47. The method of any of embodiments 42-46, wherein each arm of the plurality of arms comprises a polymeric linker segment attached to the central elastomer, the polymeric linker segment comprising polycaprolactone.

Embodiment 48. The method of embodiment 47, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), a copolymer of DL-lactide and glycolide (50/50 molar ratio), and polyethylene oxide.

Embodiment 49. The method of embodiment 48, wherein each arm of the plurality of arms comprises a first inert segment attached to the first disintegrating matrix segment, the first inert segment comprising polycaprolactone and (BiO)₂CO₃.

Embodiment 50. The method of embodiment 49, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

Embodiment 51. The method of embodiment 50, wherein each arm of the plurality of arms comprises an inactive segment attached to the second disintegrating matrix segment, the inactive segment comprising polycaprolactone, copovidone, and a poloxamer.

Embodiment 52. The method of embodiment 51, wherein a drug-eluting arm of the plurality of arms comprises the drug-eluting component attached to the inactive segment.

Embodiment 53. The method of any of embodiment 28-52, wherein wherein the area under the curve of the gastric residence system is between 1000 and 6000 hr·ng/mL.

Embodiment 54. A gastric residence system comprising: a plurality of arms affixed to a central elastomer, wherein at least one arm comprises a drug-eluting component; each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein the at least one arm comprising a drug eluting component comprises: a polymeric linker segment; a first disintegrating matrix segment attached to the polymeric linker segment; a first inert segment attached to the first disintegrating matrix segment; a second disintegrating matrix segment attached to the first inert segment; an inactive segment attached to the second disintegrating matrix segment; and the drug-eluting component attached to the inactive segment, wherein the drug eluting component comprises 50-60 wt % methadone or a salt thereof, 35-50 wt % polycaprolactone, and 0.5-3 wt % poloxamer, and wherein the drug eluting component further comprises a coating comprising a release rate-modulating polymer film.

The foregoing description sets forth exemplary systems, methods, techniques, parameters, and the like. It should be recognized, however, that such description is not intended as a limitation on the scope of the present disclosure but is instead provided as a description of exemplary embodiments.

Although the description herein uses terms first, second, etc. to describe various elements, these elements should not be limited by the terms. These terms are only used to distinguish one element from another.

1. A gastric residence system comprising:
at least one drug-eluting component comprising methadone or a salt thereof, 35-50 wt % polycaprolactone, and 0.5-3 wt % poloxamer; and
a release rate-modulating film coating the at least one drug-eluting component,
wherein the gastric residence system is configured to be maintained within a stomach of a human body for at least 48 hours and to release methadone for at least 48 hours, and the at least one drug-eluting component with the release rate-modulating film is configured to release at least 10% of the methadone or the salt thereof after the first 24 hours of residence within the stomach.

2. The gastric residence system of claim 1, wherein the at least one drug-eluting component comprises 40-70 wt % racemic methadone.

3. The gastric residence system of claim 1, wherein the at least one drug-eluting component comprises 40-70 wt % levomethadone.

4. The gastric residence system of claim 1, wherein the at least one drug-eluting component comprises 1-2 wt % poloxamer.

5. The gastric residence system of claim 1, wherein the poloxamer comprises P407.

6. The gastric residence system of claim 1, wherein the release rate-modulating film comprises polycaprolactone, copovidone, and magnesium stearate.

7. The gastric residence system of claim 6, wherein the release rate-modulating film comprises 60-90 wt % polycaprolactone.

8. (canceled)

9. The gastric residence system of claim 6, wherein the release rate-modulating film comprises 10-40 wt % copovidone.

10. (canceled)

11. The gastric residence system of claim 6, wherein the release rate-modulating film comprises 1-5 wt % magnesium stearate.

12. (canceled)

13. The gastric residence system of claim 1, wherein the at least one drug-eluting component comprises 20 mg to 50 mg of racemic methadone or a salt thereof.

14. The gastric residence system of claim 1, wherein the at least one drug-eluting component comprises 20 mg to 50 mg of levomethadone or a salt thereof.

15. The gastric residence system of claim 1, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

16. The gastric residence system of claim 15, wherein the plurality of arms comprises six arms.

17. The gastric residence system of claim 15, wherein at least two arms of the plurality of arms comprises a drug-eluting component of the at least one drug-eluting component.

18-19. (canceled)

20. The gastric residence system of claim 15, wherein each arm of the plurality of arms comprises a polymeric linker segment attached to the central elastomer, the polymeric linker segment comprising polycaprolactone.

21. The gastric residence system of claim 20, wherein each arm of the plurality of arms comprises a first disintegrating matrix segment attached to the polymeric linker segment, the first disintegrating matrix segment comprising polycaprolactone, an acid terminated copolymer of DL-lactide and glycolide (50/50 molar ratio), and polyethylene oxide.

22. The gastric residence system of claim 21, wherein each arm of the plurality of arms comprises a first inert segment attached to the first disintegrating matrix segment, the first inert segment comprising polycaprolactone and (BiO)₂CO₃.

23. The gastric residence system of claim 22, wherein each arm of the plurality of arms comprises a second disintegrating matrix segment attached to the first inert segment, the second disintegrating matrix segment comprising polycaprolactone, hydroxypropyl methylcellulose acetate succinate, and a poloxamer.

24. The gastric residence system of claim 23, wherein each arm of the plurality of arms comprises an inactive segment attached to the second disintegrating matrix segment, the inactive segment comprising polycaprolactone, copovidone, and a poloxamer.

25. The gastric residence system of claim 24, wherein a drug-eluting arm of the plurality of arms comprises the drug-eluting component attached to the inactive segment.

26. The gastric residence system of claim 1, wherein an area under the curve of the gastric residence system is between 1000 and 6000 hr·ng/mL.

27. (canceled)

28. A method of making a gastric residence system comprising:
extruding at least one drug-eluting component comprising methadone or a salt thereof, 35-50 wt % polycaprolactone, and 0.5-3 wt % poloxamer; and
applying a release rate-modulating film to the at least one drug-eluting component,
wherein the gastric residence system is configured to be maintained within a stomach of a human body for at least 48 hours and to release methadone for at least 48 hours, and the at least one drug-eluting component with the release rate-modulating film is configured to release at least 10% of the methadone or the salt thereof after the first 24 hours of residence within the stomach.

29. The method of claim 28, wherein the at least one drug-eluting component comprises 40-70 wt % racemic methadone.

30. The method of claim 28, wherein the at least one drug-eluting component comprises 40-70 wt % levomethadone.

31-39. (canceled)

40. The method of claim 28, wherein the at least one drug-eluting component comprises 20 mg to 50 mg of racemic methadone or a salt thereof.

41. The method of claim 28, wherein the at least one drug-eluting component comprises 20 mg to 50 mg of levomethadone or a salt thereof.

42. The method of claim 28, wherein the gastric residence system comprises a central elastomer and a plurality of arms, each arm of the plurality of arms comprising a proximal end affixed to the central elastomer and a distal end, wherein each arm of the plurality of arms extends radially from the central elastomer, and at least one arm of the plurality of arms comprises the at least one drug-eluting component.

43-53. (canceled)

54. A gastric residence system comprising:
a plurality of arms affixed to a central elastomer, wherein at least one arm comprises a drug-eluting component;
each arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end;
wherein the at least one arm comprising a drug eluting component comprises:
a polymeric linker segment;
a first disintegrating matrix segment attached to the polymeric linker segment;
a first inert segment attached to the first disintegrating matrix segment;
a second disintegrating matrix segment attached to the first inert segment;
an inactive segment attached to the second disintegrating matrix segment; and
the drug-eluting component attached to the inactive segment, wherein the drug eluting component comprises 40-70 wt % methadone or a salt thereof, 35-50 wt % polycaprolactone, and 0.5-3 wt % poloxamer, and wherein the drug eluting component further comprises a coating comprising a release rate-modulating polymer film.
